(19) United States
(12) Patent Application Publication
(10) Pub. No.: US 2014/0079785 A1
(43) Pub. Date: Mar. 20, 2014

(54) COMPOSITION COMPRISING LIPID NANOPARTICLES AND A CORTICOSTEROID OR VITAMIN D DERIVATIVE

(71) Applicants: Louise Bastholm Jensen, Ballerup (DK); Karsten Petersson, Ballerup (DK)

(72) Inventors: Louise Bastholm Jensen, Ballerup (DK); Karsten Petersson, Ballerup (DK)

(73) Assignee: LEO PHARMA A/S, Ballerup (DK)

(21) Appl. No.: 14/006,890

(22) PCT Filed: Mar. 23, 2012
(86) PCT No.: PCT/EP2012/055222

§ 371 Date: Oct. 15, 2013

Related U.S. Application Data
(60) Provisional application No. 61/467,192, filed on Mar. 24, 2011.

(51) Int. Cl. A61K 31/593; A61K 31/573

(52) U.S. Cl. CPC A61K 31/593; A61K 31/573
USPC 424/489; 514/180; 514/167

(57) ABSTRACT

A pharmaceutical composition comprises, as a therapeutically active ingredient, a corticosteroid and/or vitamin D derivative incorporated as a solid solution or dispersion in lipid nanoparticles, said lipid nanoparticles being solid at ambient temperature and comprising a first lipid with a melting point above body temperature, the first lipid being a wax selected from the group consisting of esters of C₁₂₋₂₄ alcohols and C₁₂₋₂₄ fatty acids, glyceryl mono-, di- or triesters of C₁₂₋₂₄ fatty acids, C₁₂₋₂₄ fatty alcohols, and cholesterol, optionally a second lipid which is an oil at ambient temperature and miscible with the first lipid, and a pharmaceutically acceptable surfactant.

### FIELD OF INVENTION

The present invention relates to a composition comprising lipid nanoparticles and a corticosteroid and/or vitamin D analogue as the active ingredient(s), a method of preparing the lipid nanoparticles and the use of the composition in the treatment of dermal diseases and conditions.

### BACKGROUND OF THE INVENTION

Atopic dermatitis (AD) is a chronically relapsing inflammatory skin disease with a high prevalence in early infancy as it affects 10-20% of all children (Katoh, J. Dermatol. 36, 2009, pp. 367-376). The disease is characterized by pruritus, erythema, lichenification, papules and dry skin with an impaired epidermal barrier and a tendency of cutaneous infections (Reitamo et al., Textbook of Atopic Dermatitis, 1^{st} Ed., Informa Healthcare, London, 2008). The pathogenesis of AD has been largely attributed to immunologic abnormalities. However, results from several recent studies demonstrate the importance of a defect skin barrier in AD as a driver for the inflammation process as well as the antigen access to the immune cells in the skin. A highly significant relationship between AD and a genetic filaggrin defect has shown that a skin barrier disruption is critical in AD pathogenesis (Palmer et al., Nat. Genet. 38, 2006, pp. 441-446; Weidinger et al., J. Allergy Clin. Immunol. 118, 2006, pp. 214-219).

Currently, the first line treatment in AD is topical corticosteroids which display a wide anti-inflammatory and immunosuppressive effect in combination with moisturizers, which can help relieve the dry skin and restore the barrier (Reitamo et al., supra). Ointments or creams with high oil content are often preferred by dermatologists because they are more effective in keeping the skin hydrated and may enhance the penetration of the drug substance applied (Wiedersberg et al., Eur. J. Pharm. Biopharm. 68, 2008, pp. 453-466; Reitamo et al., supra). However, the cosmetic acceptance of these types of formulations may be poor which is reflected in a lower compliance among the AD patients (Yentzer et al., J. Drugs Dermatol. 9, 2010, pp. 324-329). Moreover, the release from conventional vehicles such as ointments and creams and the subsequent skin penetration of the drug substance may be quite limited and involve unspecific delivery e.g. drug levels may be too low to induce a therapeutic effect in some patients while inducing adverse effects or permeating systemically in others (Korting and Schaefer-Korting, Handbook Exp. Pharmacol., 2010, 435-468). The fact that the skin barrier in AD is impaired further complicates the targeted delivery of the drug to the skin, as the barrier condition is crucial for the amount of drug substance that penetrates into and permeates across the skin (Bronaugh and Stewart, J. Pharm. Sci. 74, 1985, pp. 1062-1066; Moon and Maibach, Exogenous Dermatoses. Environmental Dermatitis (Menne and Maibach, Eds.), CRC, Boca Raton, 1991, pp. 217-226; Simonsen and Fullerton, Skin Pharmacol. Physiol. 20, 2007, pp. 230-236; Wilhelm et al., J. Invest. Dermatol. 97, 1991, pp. 927-932).

A way of optimizing the cutaneous drug delivery is the use of nanoparticulate carriers. Especially particulate carriers made from lipids (lipid nanoparticles, liposomes, micro- and nanoemulsions) are promising as they may possess occlusive properties, skin penetration enhancing and targeting properties (Korting and Schaefer-Korting, supra). Solid lipid nanoparticles (SLN) are between 40-1000 nm and in principle comparable to an o/w emulsion but the liquid oil is replaced by a lipid with a melting point above body temperature. (Muller et al., Adv. Drug Del. Review 54, Suppl. 1, 2002, S131-S155). As a result of their solid state and small size, SLN have strong adhesive properties which may induce occlusion upon application to the skin, and they are more cosmetically acceptable than an ointment (Jenning et al., Int. J. Pharm. 199, 2000, pp. 167-177; Santos et al., J. Drug Target., 2002, pp. 489-495; Wissing and Muller, Eur. J. Pharm. Biophar. 56, 2003, pp. 67-72). The occlusive properties reduce the transepidermal water loss (TEWL) and may help to physically restore the skin barrier in a skin disease like AD (Keck and Schwabe, J. Biomed. Nanotechnol. 5, 2009, pp. 428-436).

Psoriasis is a chronic inflammatory skin disease that manifests as erythematous, dry, scaling plaques resulting from hyperkeratosis. The plaques are most often found on the elbows, knees and scalp, though more extensive lesions may appear on other parts of the body, notably the lumbosacral region. The most common treatment of mild to moderate psoriasis involves topical application of a composition containing a corticosteroid as the active ingredient. While efficacious, application of corticosteroids has the disadvantage of a number of adverse effects such as skin atrophy, striae, acneiform eruptions, perioral dermatitis, overgrowth of skin fungus and bacteria, hypopigmentation of pigmented skin and rosacea.

For many years, however, an advantageous non-steroidal treatment of psoriasis has consisted in topical treatment with the vitamin D analogue compound, calcipotriol, formulated in an ointment composition (marketed as Daivonex® or Dovonex® ointment by LEO Pharma) in which the calcipotriol is present in solution or a cream composition (marketed as Daivonex® or Dovonex® cream by LEO Pharma). The solvent in the ointment composition is propylene glycol which has the advantage of enhancing penetration of the active ingredient into the skin, leading to an improved efficacy, but which is also known to act as a skin irritant. Thus, it has been reported that the inclusion of propylene glycol in topical compositions frequently causes patients to develop contact dermatitis (one study reported a number of irritant reactions to propylene glycol of 12.5%, cf. M. Hannuksela et al., Contact Dermatitis 1, 1975, pp. 112-116), and the number of irritant reactions increases when propylene glycol is used in high concentrations (as reviewed by J. Catanzaro and J. Graham Smith, J. Am. Acad. Dermatol. 24, 1991, pp. 90-95). Due to the improved penetration of calcipotriol into the skin resulting, inter alia, from the presence of propylene glycol, Daivonex® ointment has been found to be more efficacious in the treatment of psoriatic lesions than Daivonex® cream, but has also caused skin irritation in a significant proportion of psoriasis patients.

Human skin, in particular the outer layer, the stratum corneum, provides an effective barrier against penetration of microbial pathogens and toxic chemicals. While this property of skin is generally beneficial, it complicates the dermal administration of pharmaceuticals in that a large quantity, if not most, of the active ingredient applied on the skin of a patient suffering from a dermal disease may not penetrate into the viable layers of the skin where it exerts its activity. To ensure an adequate penetration of the active ingredient to the dermis and epidermis, it is generally preferred to include the active ingredient in a dissolved state, typically in the presence of a solvent in the form of an alcohol, e.g. ethanol, or diol, e.g. propylene glycol. As indicated above, propylene glycol is a well-known penetration enhancer, i.e. a substance which is capable of penetrating the stratum corneum and “draw” low-molecular components such as therapeutically active components in the vehicle into the epidermis. Propylene glycol may in itself give rise to significant skin irritation, and it is also capable of “drawing” low-molecular and potentially irritative components of the vehicle into the epidermis, leading to an overall irritative effect of conventional vehicles including propylene glycol. For this reason, the presence of propylene glycol as a solvent in compositions intended for the treatment of inflammatory skin diseases may exacerbate the inflammatory response.

An object of the invention is to provide a composition with improved penetration into the skin and improved biological activity of a corticosteroid or vitamin D analogue included in a topical composition as active ingredients compared to commercial ointments in the absence of conventional penetration enhancers such as propylene glycol or other excipients which are potential irritants. Another object is to provide a composition which possesses occlusive properties but which has improved cosmetic properties, i.e. which is less greasy than an ointment and has a more agreeable “skin feel”.

Acne is a skin condition which is a multifactorial disease affecting the pilosebaceous follicles, characterised by increased sebum production and release of sebum from the sebaceous glands, the presence of excessive amounts of sebum in the duct of the pilosebaceous follicles leading to the formation of comedones (solidified sebum plugs in the follicular duct). Further closing of the ducts results in the formation of pustules, papules or cysts which are often subject to bacterial colonisation, especially by Propionibacterium acnes, and localised inflammation. Acne vulgaris is the most common skin disorder among teenagers, but substantial numbers of adults aged 20-40 are also affected by acne. Currently available drugs for the treatment of acne include benzoyl peroxide, azelaic acid, topical and systemic antibiotics, such as Fucidin®, clindamycin, erythromycin, and tetracyclin, retinoids, such as adapalene, tretinoin, isotretinoin, and hormones, such as estrogen. There are, however, serious drawbacks with these medications including teratogenicity, skin irritation, photosensibilisation, etc.

Because of the negative psychosocial consequences for affected individuals, and the relatively limited numbers of drugs available for topical treatment of acne and the severity of the known side effects of these drugs, the provision of new medicaments for adequate therapy of acne is very important.

Thus, it is a further object of the invention to provide a composition capable of targeting an active ingredient included therein to hair follicles in skin. Follicular targeting is of particular interest in case of compositions intended for the treatment of acne and related disorders such as rosacea. Targeting the active ingredient directly to the site of action may have the added advantage of reducing adverse effects such as skin irritation so as to provide a therapy for acne which is better tolerated than the treatment options currently on the market.

### SUMMARY OF THE INVENTION

The skin penetration of a drug substance intended for use locally in the skin is a complex process involving three major steps: 1) release of the substance from the vehicle, 2) penetration into the stratum corneum and 3) partitioning from stratum corneum to target sites in viable epidermis and dermis. The first step is dependent on the physicochemical properties of the drug and the vehicle which may be optimized by the processing. The second and third steps are more complex. Again, the physicochemical properties of the drug substance and the degree of drug saturation in the vehicle are important for the partitioning of the drug substance between the vehicle and the skin, and may be affected by optimization of the vehicle. But in addition, the condition of the major biological barrier for penetration into the skin—the stratum corneum—is influenced by skin diseases. In the research leading to the present invention, lipid nanoparticles were evaluated for their properties as a topical drug delivery system for barrier-impaired skin. The lipid nanoparticles were compared with a conventional ointment formulation which is suitable for delivery of lipophilic compounds to the skin and possesses occlusive properties.

Recently, in vitro studies with barrier-impaired skin showed that SLN were able to retain a significantly higher level of a corticosteroid in the skin as compared to an ointment. This reservoir effect was seen for both intact and barrier-impaired skin in vitro and the drug was localized distinctively in the stratum corneum. The correlation between the specific localization of the drug substance in the upper skin layers, the skin reservoir effect, and the therapeutic effect of the drug applied to the skin after application of SLN containing the drug is poorly described. It is an object of the present invention to provide a composition with increased drug efficacy as a result of the ability of lipid nanoparticles to occlude the skin or to increase the interaction with the barrier-impaired skin.

Accordingly, the present invention relates to a topical pharmaceutical composition comprising, as a therapeutically active ingredient, a corticosteroid incorporated as a solid solution or dispersion in lipid nanoparticles, said lipid nanoparticles being solid at ambient temperature and comprising about 60-92% by weight of a first lipid with a melting point above body temperature, said first lipid being a wax selected from the group consisting of esters of C₁₂₋₂₄ alcohols and C₁₂₋₂₄ fatty acids, glyceryl mono-, di- or triesters of C₁₂₋₂₄ fatty acids, C₁₂₋₂₄ fatty alcohols, and cholesterol, said lipid nanoparticles further comprising about 2-25% by weight of a pharmaceutically acceptable surfactant.

In another aspect, the invention relates to a topical pharmaceutical composition comprising, as a therapeutically active ingredient, a vitamin D derivative incorporated as a solid solution or dispersion in lipid nanoparticles, said lipid nanoparticles being solid at ambient temperature and comprising about 60-92% by weight of a first lipid with a melting point above body temperature, said first lipid being a wax selected from the group consisting of esters of C₁₂₋₂₄ alcohols and C₁₂₋₂₄ fatty acids, glyceryl mono-di- or triesters of C₁₂₋₂₄ fatty acids with an acid value of 0.1 or less, C₁₂₋₂₄ fatty alcohols, and cholesterol, said lipid nanoparticles further comprising about 2-25% by weight of a pharmaceutically acceptable surfactant selected from the group consisting of poloxamers or ethoxylated fatty alcohols.

### BRIEF DESCRIPTION OF THE DRAWINGS

The invention is further described in the appended drawings.

FIG. 1 shows the relative amount of ³H BMV penetrated into the different skin layers after applying BMV in an ointment and the three different SLN, respectively, onto intact (a) and barrier-impaired (b) skin for 24 h. The results are relative to the total amount of ³H BMV recovered. Mean±SD (n=8).

FIG. 2 shows the relative amount of ³H BMV penetrated into the skin and into the receptor medium, respectively, after applying BMV in an ointment and distearate SLN, respectively, onto intact (a) and barrier-impaired (b) skin for 6, 16 or 24 h. The results are relative to the total amount of ³H BMV recovered. Mean±SD (n=6).

FIG. 3 shows the relative amount of ³H BMV in the skin (a) and in the receptor medium (b) after application of distearate SLN and ointment, respectively, for 24 h. The skin exposed to SLN was in some cases occluded after application of SLN. The results are relative to the total amount of ³H BMV recovered. Mean±SD (n=8).

FIG. 4 shows the skin concentration of BDP in the skin of hairless rats treated with BDP in SLN composed of glyceroldistearate compared with BDP in an ointment. The SLN composition is shown in Example 1.

FIG. 5a shows the AUC of ear thickness in the study which compared the effect of BMV administered in SLN with BMV in an ointment. Mean±SEM (n=10).

5b shows the AUC of ear thickness in the dose finding-study. Mean±SEM (n=8). All animals were treated with oxazolone according to the treatment scheme described in Example 3 in order to induce the AD phenotype.

FIGS. 6a and 6b show the IL-4 and Il-1βcytokine levels in sensitized mice. All animals were treated with oxazolone according to the treatment scheme described in Example 3 order to induce the AD phenotype. Mean±SEM (n=4-10).

FIGS. 7a and 7b are graphs showing the reduction of transepidermal water loss (TEWL) by application of lipid nanoparticles containing BMV on the ears of oxazolone treated mice compared to application of an ointment containing BMV. FIG. 7a shows a comparison of SLN with ointment. FIG. 7b shows TEWL values from a dose-finding study.

### DETAILED DISCLOSURE OF THE INVENTION

### Definitions

The term “lipid nanoparticles” is intended to mean either solid lipid nanoparticles (SLN) or nanostructured lipid carriers (NLC). SLN are nanosized particles prepared from lipids that are solid at ambient temperature (e.g. long-chain triglycerides, fatty acids and waxes) and surfactants, in which an active ingredient may be dissolved or dispersed. NLC are nanosized particles prepared from lipids that are solid at ambient temperature and lipids that are liquid at ambient temperature (oils) and surfactants, in which an active ingredient may be dissolved or dispersed. The particle size of the lipid nanoparticles may be in the range of about 10-800 nm, such as 50-600 nm or 100-500 nm.

The term “active ingredient” is intended to indicate a therapeutically active drug substance selected from vitamin D derivatives or analogues and corticosteroids.

The term “vitamin D derivative” is intended to indicate a biologically active metabolite of vitamin D₃, such as calcitriol, or a precursor to such a metabolite, such as alfacalcidol.

The term “vitamin D analogue” is intended to indicate a synthetic compound comprising a vitamin D scaffold with sidechain modifications and/or modifications of the scaffold itself. The analogue exhibits a biological activity on the vitamin D receptor comparable to that of naturally occurring vitamin D compounds.

“Calcipotriol” is a vitamin D analogue of the formula

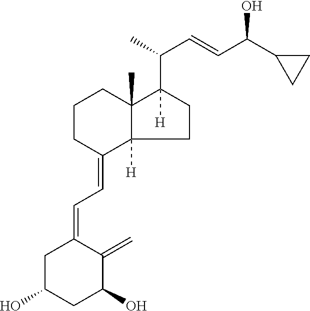

Calcipotriol has been found to exist in two crystalline forms, an anhydrate and a monohydrate. Calcipotriol monohydrate and its preparation are disclosed in WO 94/15912.

The term “corticosteroid” is intended to indicate steroid compounds synthesized in the adrenal cortex from cholesterol or derivatives thereof, in particular such derivatives that exert an anti-inflammatory effect. Examples of corticosteroids include, but are not limited to, of amcinonide, betamethasone, budenoside, clobetasol, clobetasone, cortisone, desonide, desoxycortisone, desoximethasone, dexamethasone, diflucortolon, diflorasone, flucortisone, flumethasone, flunisolide, fluocinonide, fluocinolon, fluorometholone, fluprednisolone, flurandrenolide, fluticasone, halcinonide, halobetasol, hydrocortisone, meprednisone, methylprednisone, mometasone, paramethasone, prednicarbate, prednisone, prednisolone and triamcinolone or a pharmaceutically acceptable ester or acetonide thereof. Examples of currently favoured corticosteroids are betamethasone and esters thereof such as the 17-valerate or 17,21-dipropionate, or clobetasol 17-propionate.

The term “storage stability” or “storage stable” is intended to indicate that the composition exhibits chemical and physical stability characteristics that permit storage of the composition for a sufficient period of time at refrigeration or, preferably, room temperature to make the composition commercially viable, such as at least 12 months, in particular at least 18 months, and preferably at least 2 years.

The term “chemical stability” or “chemically stable” is intended to mean that no more than 10%, preferably no more than 6%, of the active ingredients degrades over the shelf-life of the product, typically 2 years, at room temperature. An approximation of chemical stability at room temperature is obtained by subjecting the composition to accelerated stability studies at 40° C. where the composition is placed in a heating cupboard at 40° C. and samples are taken at 1 and 3 months and tested for the presence of degradation products by HPLC. If less than about 10% of the substance has degraded after 3 months at 40° C., this is usually taken to correspond to a shelf-life of 2 years at room temperature. When the active ingredient included in the composition is calcipotriol, “chemical stability” usually indicates that the calcipotriol does not degrade significantly over time to 24-epi calcipotriol or other degradation products of calcipotriol in the finished pharmaceutical product.

The term “physical stability” or “physically stable” is intended to mean that the active ingredients do not precipitate from the propellant or vehicle phases over the shelf life of the composition.

The term “substantially anhydrous” is intended to mean that the content of free water in the ointment composition does not exceed about 2% by weight, preferably not about 1% by weight, of the composition.

The term “medium-chain triglycerides” is used to indicate triglyceride esters of fatty acids with a chain length of 6-12 carbon atoms. A currently favoured example of such medium chain triglycerides is a mixture of caprylic (C₈) and capric (C₁₀) triglycerides, e.g. available under the trade name Miglyol 812.

The term “semi-solid” is used to denote a composition or excipient which shows viscoelastic behaviour and is non-Newtonian in character, i.e. does not flow at low shear stress, but exhibits plastic, pseudoplastic or thixotropic flow behaviour at high shear rates at room temperature. Typical examples of semi-solid compositions are ointments and creams.

The term “occlusive” is intended to indicate the provision of a lipid layer on the skin surface which forms a hydration barrier sufficient to result in reduction of transepidermal water loss, resulting in skin hydration.

The term “skin penetration” is intended to mean the diffusion of the active ingredient into the different layers of the skin, i.e. the stratum corneum, epidermis and dermis.

The term “skin permeation” is intended to mean the flux of the active ingredient through the skin into the systemic circulation or, in case of in vitro studies such as those reported in Example 2 below, the receptor fluid of the Franz cell apparatus used in the experiment.

### Embodiments

In the present composition, the first lipid may comprise about 65-92% by weight, or about 70-90% by weight, or about 75-85% by weight, or about 80% by weight, of the lipid nanoparticles, and the surfactant comprises about 8-22% by weight, such as about 10-20% by weight, of the lipid nanoparticles. The first lipid may favourably be selected from the group consisting of cetylpalmitate, beeswax, stearyl palmitate, stearyl behenate, glycerol monostearate, glycerol distearate, glycerol dibehenate, glycerol trimyristate, glycerol tripalmitate, glycerol tristearate, behenol, stearic acid, hydrogenated palm oil, hydrogenated coco-glycerides, hydrogenated castor oil or cetostearylalcohol

In the present composition, the surfactant may be a hydrophilic surfactant and may favourably be selected from the group consisting of poloxamers such as Poloxamer 188 or Poloxamer 407, polysorbates such as polysorbate 80, sugar esters (such as sucrose stearate or sucrose palmitate), ethoxylated fatty alcohols such as polyoxyethylene castor oil derivatives or it may be a lipophilic surfactant such as a phospholipid such as soy phosphatidylcholine or egg lecithine.

In addition, the lipid nanoparticles may comprise about 1-40%, such as about 10-30% by weight or about 15-15% by weight or about 20% by weight, of the lipid nanoparticles of a second lipid which is an oil at ambient temperature and miscible with the first lipid, or a lipophilic emulsifier (e.g. a polyoxypropylene fatty acyl ether) or emollient. The second lipid may be selected from the group consisting of a C₆₋₁₀ monoglyceride, C₆₋₁₀ diglyceride, isopropyl myristate or isopropyl palmitate, medium chain triglycerides or long chain triglycerides, including vegetable oils such as castor oil, sunflower oil, safflower oil, evening primrose oil, borage seed oil, sesame oil, corn oil, palm kernel oil, olive oil, avocado oil, almond oil, rapeseed oil, coconut oil, cottonseed oil, peanut oil, soybean oil, wheat germ oil, grape kernel oil or jojoba oil.

The present composition may be a cream (oil-in-water emulsion), lotion, sprayable formulation or aqueous gel wherein the lipid nanoparticles are typically present in an amount of about 1-40% by weight, such as about 5-30% by weight or about 10-20% by weight of the composition. The composition further comprises an aqueous phase in which the lipids may, under certain circumstances, form a network so as to cause gelling of the composition, or which may contain a thickener. The thickener may be selected from the group consisting of a carbomer, a cellulose derivative such as hydroxypropylcellulose, hydroxypropylmethylcellulose, hydroxyethylcellulose, hyaluronic acid, alginate, dextran or a derivative thereof. The thickener is typically present in an amount of about 0.1-5% by weight, such as about 0.5% by weight, of the composition. The composition may further comprise an emollient which may be selected from the group consisting of silicone oil, liquid paraffin and cholesterol or glycerol, allantoin, panthenol, polyglycerol or a polyglycerol ester. The emollient may be included in an amount of about 10-50% by weight, or about 20-40% by weight, or about 30% by weight of the composition.

The active ingredient may be present in the composition in different forms, i.e. dissolved or dispersed in the aqueous phase, dissolved or dispersed in the surface layer lipid/aqueous phase or dissolved or dispersed in the lipid nanoparticles, either on the surface or in the core. It has previously been found that the active ingredient is most likely associated with the lipid surface and not efficiently incorporated in the core, causing zero order release profiles and lack of specific skin targeting after 6 h [L. B. Jensen et al., Int. J. Pharm. 390(1), 2010, pp. 53-60]. This is in agreement with the present findings according to which some of the active ingredient is dissolved or dispersed in the aqueous phase, at least in cetylpalmitate and tripalmitate lipid nanoparticles, while the major proportion of the active ingredient is incorporated in or associated with the lipid surface. It is generally believed that a higher solubility in the lipid component and a more efficient encapsulation of the active ingredient may be achieved with a more polar lipid compared to a less polar lipid J. Zhang and E. Smith, J. Pharm. Sci. DOI 10.1002/jps.22329, 2010]. Thus, solubility studies with betamethasone valerate (BMV, a model active ingredient) in the melted lipid showed a correlation between lipid polarity and the solubility in the vehicle, corresponding to previous findings with lipids containing varying amounts of monoglycerides [L. B. Jensen et al., supra; V. V. Kumar et al., Int. J. Pharm. 335(1-2), 2007, pp. 167-175]. Lipid polarity has been shown to control the release of BMV in in vitro release studies conducted with different lipids with varying polarity [L. B. Jensen et al., supra].

The effect of lipid polarity could also be relevant for the skin penetration of active ingredients formulated in lipid nanoparticles, may be as a result of a changed interaction of the lipid nanoparticles with skin lipids. Components with a solubility parameter close to that of the skin may have good miscibility with the skin, and varying the lipid solubility parameter can be a way of controlling the release of the active ingredient from the lipid nanoparticles and partitioning into the skin [K. B. Sloan et al., J. Invest. Dermatol. 87(2), 1986, pp. 244-252]. By way of example, the solubility parameter for BMV is estimated to be 12 [L. B. Jensen et al., supra] and around 10 for porcine skin [Z. Liron and S. Cohen, J. Pharm. Sci. 73(4), 1984, pp. 538-542]. The lipids used had solubility parameters between 8.7 and 9.5. Lipid nanoparticles composed of distearate, which has a solubility parameter of 9.5, closest to that of the skin, would thus be expected to be more miscible with the skin lipids than tripalmitate and cetylpalmitate lipid nanoparticles. BMV was soluble in this lipid and distearate lipid nanoparticles were smaller in size than the other lipid nanoparticles applied; the diameter was 150.9±0.12 nm. This may explain the higher amount of BMV in the skin and particularly in the stratum corneum after application of BMV in distearate lipid nanoparticles as compared to tripalmitate and cetylpalmitate lipid nanoparticles (cf. Example 2).

To minimize any differences in penetration profiles caused by differences in size, it was the intention that the lipid particles should not differ significantly in their mean diameters. The mean diameter is correlated to the particle surface area, which is thought to be an important parameter for the interaction of the lipid nanoparticles with and penetration into the skin as well as any occlusive properties [S. Wissing et al., J. Cosmet. Sci. 52(5), 2001, pp. 313-324; R. H. Muller et al., Adv. Drug Deliv. Rev. 54, Suppl. 1, 2002, S131-S155]. In the present composition, the lipid nanoparticles may therefore have a mean diameter in the range of about 10-800 nm, in particular about 50-600 nm, such as 100-500 nm. The mean diameter of the exemplified BMV lipid nanoparticles (cf. Example 1) was in the range of 150-212 nm, with a PdI of less than 0.24. It is generally believed that particles above 10 nm do not penetrate intact skin but that diseased skin may be penetrated by larger sized particles (with a diameter of 500-700 nm) because the barrier is impaired. The measured particle size indicates that the particles may not be able to penetrate the intact skin, whereas the barrier-impaired skin may be prone to particle penetration. Lipid nanoparticles made from cetylpalmitate were more monodisperse than the other types of lipids, which may be due to cetylpalmitate being a wax type lipid with a different crystal structure. This type of lipid has previously been shown to result in homogenous and small sized particles with excellent physical stability [V. Jenning and S. Gohla, Int. J. Pharm. 196(2), 2000, pp. 219-222].

The size measurements indicated that SLN could not penetrate the intact skin, but for barrier impaired skin, particle penetration was possible. The studies discussed in Example 2 below with ¹⁴C-labelled lipid particles and ³H-labelled BMV showed that the ratio of BMV/lipid increased down the skin layers to reach a maximum in the receptor medium, and for this reason it was concluded that BMV was not delivered to the target cells in the deeper skin layers by the lipid nanoparticles, which rather serve as a drug reservoir of drug substance in the upper layers of the skin. From the ¹⁴C-labelling of the lipid, it was also shown that the lipid particles mainly stay on the skin surface and in the stratum corneum in both intact and barrier-impaired skin. Thus, the active ingredient must be released from the particles to diffuse further into the skin and reach the target cells in the viable epidermis and dermis.

It is considered beneficial that the lipid nanoparticles do not permeate across the skin, even if the barrier is strongly impaired as illustrated here by tape-stripped skin. The fact that the lipid particles stay on the surface of the skin also makes it probable that lipid nanoparticles may improve skin hydration and physically strengthen the barrier. This is a highly relevant property in the treatment of a skin disease such as AD, where the physical skin barrier condition is strongly related to the AD pathogenesis as it interacts with the immune skin barrier.

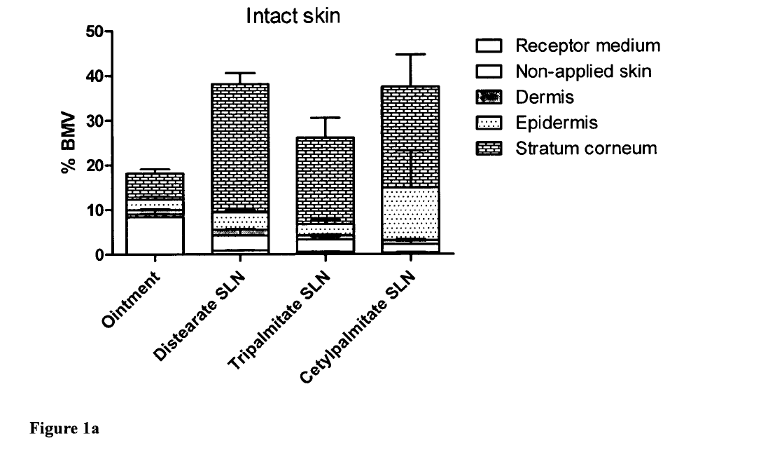

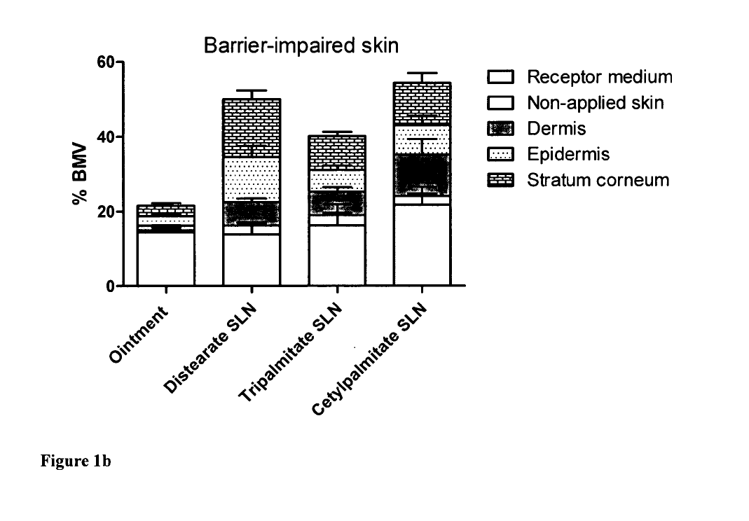

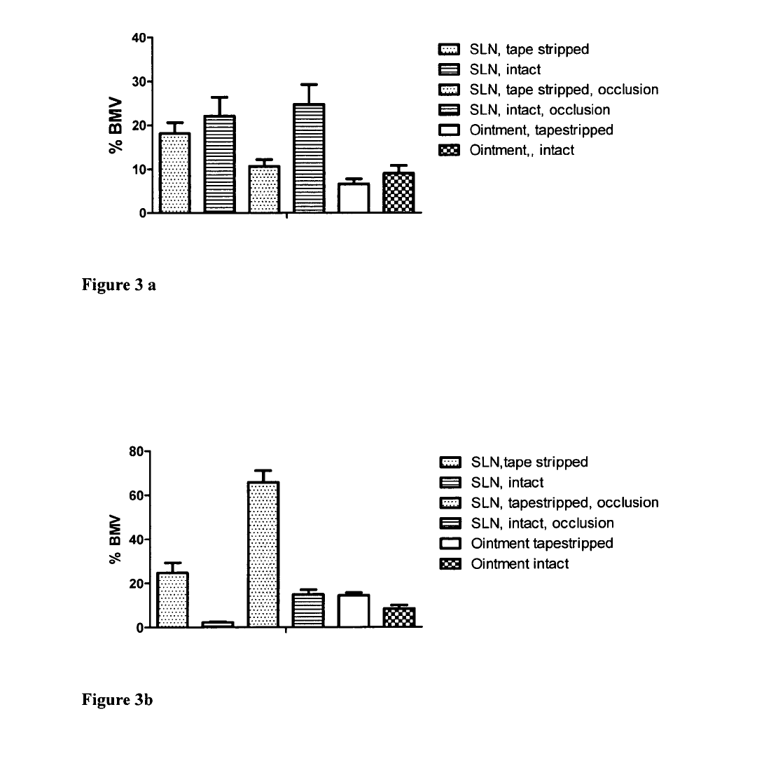

Lipid nanoparticles were superior to the ointment in achieving a high amount of drug substance in the skin. This may be clearly seen from the results in FIG. 3. A large proportion of the active ingredient was found in the upper layer of the skin, intact and barrier impaired, which was most likely to be related to the large surface area and adhesive properties of the lipid nanoparticles. In addition, the lipid nanoparticles and active ingredient may penetrate hair follicles and skin furrows from where they can act as a drug reservoir. After application of distearate lipid nanoparticles to intact and barrier-impaired skin, a higher amount of the active ingredient was present in the stratum corneum and epidermis compared to tripalmitate and cetylpalmitate, which is thought to be related to differences in lipid polarity and particle size (FIG. 1). Thus, the close interaction with the skin, the fusion with the skin lipids and the release of the drug substance in a controlled manner can be the mechanism of the penetration-enhancing properties of the lipid nanoparticles, besides the occlusive effect seen before [R. H. Muller et al., supra].

The higher amount of BMV in the epidermis of intact skin and in the receptor medium for intact and barrier-impaired skin when applying cetylpalmitate SLN can be attributed to the different lipid structure, lower polarity and lower BMV solubility [J. Zhang and E. Smith, supra; V. Jenning and S. Gohla, supra]. This may cause BMV to leave the SLN vehicle more readily and partition into the skin.

BMV was retained very efficiently in the skin when the barrier was intact (FIG. 1a) but permeated across the skin similarly to the ointment when the barrier was impaired (FIG. 1b). However, a higher amount of the active ingredient was found in barrier-impaired skin compared to the ointment. The most favourable penetration profile of the tested lipid nanoparticles (cf. Example 2) was obtained with distearate lipid nanoparticles which resulted in the highest solubility of the active ingredient and were superior in retaining a high amount of active ingredient in the skin and a lower amount of active ingredient in the receptor medium. The level of active ingredient in the skin was similar for intact and barrier-impaired skin, which indicates that the lipid nanoparticles adhered effectively to the surface of the intact skin as well as the barrier-impaired skin.

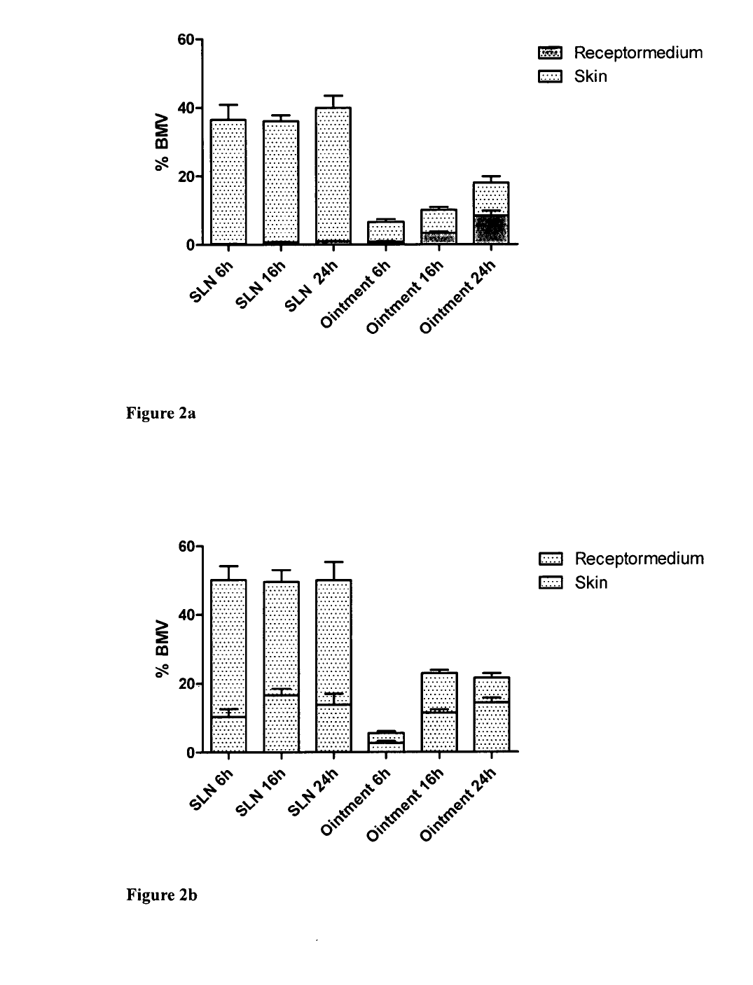

It has been found that a higher amount of the active ingredient was retained in the skin, intact as well as impaired, during application of distearate SLN for 6, 16 and 24 h, compared to an ointment (FIG. 2). The minimal increase in the receptor medium during the 24 h can be explained by surface depletion of the formulation, i.e. the amount of active ingredient available for penetration into the skin. The active ingredient is believed to be partly incorporated in the particles, partly in the particle surface layer. This structure has previously been shown to be reflected in a burst release followed by a controlled release [E. B. Souto et al., Int. J. Pharm 278(1), 2004, pp. 71-77; V. Jenning et al., J. Control. Release 66(2-3), 2000, pp. 115-126]. When the surface layer is depleted upon burst release, then less of the active ingredient may diffuse from the particles to the skin and with a slower rate of release. Another reason for the profile seen is saturation of the skin or the hair follicles in particular, either by the BMV incorporated in the SLN or by any BMV crystals, which were present in SLN made from cetylpalmitate and tripalmitate, respectively. An initial transient shunt diffusion through hair follicles and sweat ducts followed by a steady state diffusion through the SC may be possible (R. J. Scheuplein, Mechanism of percutaneous absorption. II. Transient diffusion and the relative importance of various routes of skin penetration, J Invest Dermatol 48 (1) (1967) 79-88). This effect may correspond to the constant level of BMV seen in these studies. Thus, lipid nanoparticles may be used to create a reservoir in the skin as well as in the hair follicles and keep a constant amount of active ingredient available for absorption. However, the rate of exchange between particles and skin may change with time of application because of the drug incorporation. The time profiles and the amount of drug substance in the receptor medium indicate that the drug substance is found in the surface layer of the particles. When applied to the skin, the lipid nanoparticles thus have the ability to deliver the active ingredient in a biphasic manner—initiated by a burst release from the surface of the particles and the aqueous phase followed by a reservoir effect in the stratum corneum from the drug substance associated more closely with the lipid particles.

The significant increase in penetration of BMV upon occlusion of barrier-impaired skin may have different reasons. First, occlusion almost always enhances the amount of drug substance absorbed in the skin because of the increased diffusion coefficient due to the increased water content in the stratum corneum and the disturbance of the lipid barrier [J. A. Bouwstra et al., J. Recept. Signal Transduct. Res. 21(2-3), 2001, pp. 259-286]. Furthermore, the occlusion may promote a penetration-enhancing effect on the skin barrier of the surfactant present in the lipid nanoparticles. Occlusion may also affect the exchange of drug between the composition and skin as the water was not allowed to evaporate after application. Because of the very large amount of drug permeating to the receptor medium upon occlusion of the barrier-impaired skin, these results also support that the active ingredient is associated with the particle surface rather than encapsulated in the solid particle core.

In another study betamethasone dipropionate (BDP) incorporated in SLN was applied to the skin of hairless rats and the ability of SLN to retain the corticosteroid to the skin was demonstrated. When compared to an ointment a significantly higher amount of BDP was present in the skin after 1 and 3 days, respectively and 7 days after the formulations were applied the drug substance could still be detected in the skin when administered in SLN but no drug substance could be detected when administered in an ointment.

Lipid nanoparticles of the type disclosed above may be prepared by a method comprising
(a) solubilizing or dispersing a corticosteroid in an lipid phase comprising about 60-92% by weight of a first lipid with a melting point above body temperature, said first lipid being a wax selected from the group consisting of esters of C₁₂₋₂₄ alcohols and C₁₂₋₂₄ fatty acids, glyceryl di- or triesters of C₁₂₋₂₄ fatty acids, C₁₂₋₂₄ fatty alcohols, and cholesterol, said first lipid being in a molten state, said lipid phase optionally comprising about 1-40% by weight of a second lipid which is an oil at ambient temperature and miscible with the first lipid, or a lipophilic emulsifier or emollient;
(b) dispersing the lipid phase obtained in step (a) in an aqueous phase comprising 0.2-10% by weight of a pharmaceutically acceptable surfactant selected from the group consisting of a poloxamer, a polysorbate and an ethoxylated fatty alcohol to form an emulsion,
(c) homogenizing the emulsion obtained in step (b) using a high-pressure homogenizer to form nanoparticles of the lipid phase comprising the corticosteroid and
(d) cooling the homogenized emulsion obtained in step (c) to solidify the nanoparticles.

The particle size of the lipid nanoparticles prepared by this method was determined to be in the range of about 100-500 nm.

Alternatively, the lipid nanoparticles may be prepared by a method comprising
(a) solubilizing or dispersing a corticosteroid in an lipid phase comprising about 60-92% by weight of a first lipid with a melting point above body temperature, said first lipid being a wax selected from the group consisting of esters of C₁₂₋₂₄ alcohols and C₁₂₋₂₄ fatty acids, glyceryl di- or triesters of C₁₂₋₂₄ fatty acids, C₁₂₋₂₄ fatty alcohols, and cholesterol, said first lipid being in a molten state, said lipid phase optionally comprising about 1-40% by weight of a second lipid which is an oil at ambient temperature and miscible with the first lipid, or a lipophilic emulsifier or emollient;
(b) solidifying the lipid phase obtained in step (a) in liquid nitrogen or on dry ice;
(c) milling the solidified lipid phase to a particle size of approximately 50-100 μm;
(d) dispersing the powder obtaining in step (c) in a cold aqueous phase containing about 0.2-10% by weight of a surfactant, and
(e) homogenizing the dispersion obtained in step (d) using a high-pressure homogenizer at room temperature or below room temperature to obtain the lipid nanoparticles.

Lipid nanoparticle compositions comprising a corticosteroid may favourably be used in the treatment of inflammatory skin diseases or conditions such as eczema, atopic dermatitis, contact dermatitis, psoriasis, skin ageing, photoageing, acne, urticaria or pruritis.

In another embodiment, the lipid nanoparticles further comprise a second lipid which is an oil at ambient temperature and miscible with the first lipid, or a lipophilic emulsifier or emollient. The amount of the second lipid is about 1-40% by weight, such as about 10-35% by weight, or about 15-30% by weight, or about 20-25% by weight of the lipid nanoparticles. More specifically, the lipid nanoparticles may comprise about 80-85% by weight of the first lipid and about 15-20% by weight of the second lipid.

In this embodiment, the second lipid may advantageously be selected from the group consisting of C₆₋₁₀ monoglycerides, C₆₋₁₀ diglycerides, medium chain triglycerides, such as caprylic/capric triglycerides, long-chain triglycerides, such as castor oil, isopropyl myristate or isopropyl palmitate.

In an embodiment comprising a vitamin D derivative or analogue as the active ingredient, said first lipid may favourably be selected from the group consisting of cetylpalmitate, a C₁₄₋₂₈ fatty alcohol, hydrogenated palm oil and a triglyceride with an acid value of 0.1 or less, indicating a low content of free fatty acids that are detrimental to the chemical stability of vitamin D derivatives due to the well-known acid sensitivity of vitamin D derivatives. In that embodiment, lipid nanoparticles comprising a first and second lipid have been found particularly favourable for the formulation of vitamin D derivatives as it permits the drug compound to be incorporated in the lipid nanoparticles where it is less exposed to degradation by deleterious components in the aqueous phase of the composition. Using a first and second lipid may increase the drug load in the lipid nanoparticles and prevent the active ingredient from being present in the aqueous phase. Furthermore when the lipid nanoparticles possess a structure which is not prone to polymorphic inversion during storage leading to drug expulsion and partitioning to the aqueous phase the stability may further increase. The second lipid may favourably be selected from isopropyl myristate, isopropyl palmitate, medium chain triglycerides such as capryl/capric triglycerides or long-chain triglycerides such as castor oil. In a currently favoured embodiment, said first lipid is cetylpalmitate and said second lipid is caprylic/capric triglyceride. The vitamin D derivative may be selected from calcipotriol, calcitriol, maxacalcitol, tacalcitol all of which are well-known for topical application on skin.

Compositions comprising drug-containing lipid nanoparticles intended for targeting to hair follicles may advantageously comprise lipids that have a solubility parameter close to that of sebum present in the pilosebaceous glands of hair follicles. Thus, it would appear that the effect of the first and/or second lipid present in the lipid nanoparticles on delivery of the active compound into sebum-rich areas such as hair follicles, may be explained by the solubility properties of the excipient, as determined by Hildebrand solubility coefficients (J H Hildebrand and R L Scott, The Solubility of Non-Electrolytes, Reinhold, N.Y., 1949). The Hildebrand coefficients (solubility parameters δ) for model sebum compositions show that sebum is an overall non-polar, oily material with a Hildebrand coefficient of about 7.5-8 (cal/cm³)^{1/2} (cf. D W Osborne et al., “The Influence of Skin Surface Lipids on Topical Formulations” in Topical Drug Delivery Formulations, Vol. 42, 1990 (Drugs and the Pharmaceutical Science)). It has been found that lipids with Hildebrand coefficients within ±2 units of that of sebum are miscible with sebum and therefore suitable for the purpose of solubilising active compounds for delivery thereof to the pilosebaceous unit. Another influencing factor will be the lipid melting point as the release of the drug substance may be initiated by melting as well as by fusion with the sebum and skin lipids. Lipids with a solubility parameter close to that of the sebum may include cetylpalmitate, stearyl palmitate and stearyl behenate.

Calcipotriol is known to be a substance which is extremely sensitive to acidic conditions (at a pH below about 7.0 in aqueous compositions or in the presence of acidic reacting substances in non-aqueous compositions) which contribute to the rapid degradation of calcipotriol. To ensure an adequate chemical stability of the substance throughout the shelf-life of the composition, it may be advisable to include a compound capable of neutralizing acidic impurities which may be present in one or more of the excipients of the composition and which are detrimental to the chemical stability of calcipotriol. The acid neutralizing compound may favourably be selected from a buffer such as a phosphate buffer which may be included in an amount of about 0.025-0.1% by weight of the composition such that the pH of the composition is 7.5 or more. The acid neutralizing compound may also be a an amine with lipid anchoring, i.e. a long lipid chain anchored in the interface between the aqueous phase and the lipid nanoparticle. Examples of such amines are tertiary amines such as triethanolamine, trometamol, monoethanolamine or diethanolamine, which may be included in the composition in an amount of about 0.1-2% by weight.

In this embodiment of the present composition, the lipid nanoparticles may be present in an amount of 1-40% by weight, such as about 5-30% by weight or about 10-20% by weight, of the composition. The composition may be a cream (oil-in-water), lotion, sprayable formulation or aqueous gel, the composition further comprising an aqueous phase. The aqueous phase may further comprises a lipid such as silicone oil, liquid paraffin or cholesterol and/or a thickener. The thickener may be selected from a carbomer, or a cellulose derivative, such as hydroxypropylcellulose, hydroxypropylmethylcellulose, hydroxyethylcellulose. The thickener is typically present in an amount of 0.1-5% by weight, in particular about 0.5% by weight, of the composition. The composition may further comprise an emollient which may be selected from the group consisting of silicone oil, liquid paraffin and cholesterol. The emollient may be included in an amount of about 10-50% by weight, or about 20-40% by weight, or about 30% by weight of the composition.

The composition according to this embodiment may be prepared by a method comprising
(a) solubilizing or dispersing a vitamin D derivative in an lipid phase comprising about 60-92% by weight of a first lipid with a melting point above body temperature, said first lipid being a wax selected from the group consisting of esters of C₁₂₋₂₄ alcohols and C₁₂₋₂₄ fatty acids, glyceryl triesters of C₁₂₋₂₄ fatty acids with an acid value of 0.1 or less, C₁₂₋₂₄ fatty alcohols, and cholesterol, and optionally about 1-40% by weight of a second lipid which is an oil at room temperature and miscible with the first lipid, said first lipid being in a molten state,
(b) dispersing the lipid phase obtained in step (a) in an aqueous phase comprising about 0.2-10% by weight of a pharmaceutically acceptable surfactant selected from a poloxamer or an ethoxylated fatty alcohol,
(c) homogenizing the emulsion obtained in step (b) using a high-pressure homogenizer to form nanoparticles of the lipid phase comprising the vitamin D derivative and
(d) cooling the homogenized emulsion obtained in step (c) to solidify the nanoparticles.

The particle size of the lipid nanoparticles prepared by this method was determined to be in the range of about 100-500 nm.

Alternatively, the composition may be prepared by a method comprising
(a) solubilizing or dispersing a vitamin D derivative in an lipid phase comprising about 60-92% by weight of a first lipid with a melting point above body temperature, said first lipid being a wax selected from the group consisting of esters of C₁₂₋₂₄ alcohols and C₁₂₋₂₄ fatty acids, glyceryl triesters of C₁₂₋₂₄ fatty acids with an acid value of 0.1 or less, C₁₂₋₂₄ fatty alcohols, and cholesterol, and optionally about 1-40% by weight of a second lipid which is an oil at room temperature and miscible with the first lipid, said first lipid being in a molten state,
(b) solidifying the lipid phase obtained in step (a) in liquid nitrogen or on dry ice;
(c) milling the solidified lipid phase to a particle size of approximately 50-100 μm;
(d) dispersing the powder obtaining in step (c) in a cold aqueous phase containing about 0.2-10% by weight of a surfactant, and
(e) homogenizing the dispersion obtained in step (d) using a high-pressure homogenizer at room temperature or below room temperature to obtain the lipid nanoparticles.

Lipid nanoparticle compositions comprising a vitamin D derivative or analogue may be used in the prevention or treatment of an inflammatory or hyperproliferative skin condition such as psoriasis, sebopsoriasis, pustulosis palmoplantaris, dermatitis, ichtyosis, rosacea, acne or actinic keratosis.

In the literature it has been described that sebocytes express the vitamin D receptor (VDR) and that expression of VDR is induced by treatment with the biologically active vitamin D metabolite 1α,25-dihydroxyvitamin D₃ (calcitriol), cf. C. Krämer et al, J. Steroid Biochem. Mol. Biol. 113, 2009, pp. 9-16. Incubation of cultured sebocytes with calcitriol resulted in reduced secretion of the proinflammatory cytokines IL-6 and IL-8. It may therefore be assumed that vitamin D derivatives may exert a therapeutic effect on disorders of the sebaceous glands such as acne, if targeted to the hair follicles. Furthermore, vitamin D derivatives are known to reduce hyperproliferation of keratinocytes and may be expected to exert that effect on keratinocytes of the follicular duct as well.

While it has previously been suggested that vitamin D analogues such as calcipotriol may be used in the treatment of acne (cf. WO 91/12807), the compositions disclosed in the prior reference were subsequently shown to be non-efficacious for such treatment. The lack of efficacy seen with the prior compositions is believed to be caused by the active ingredient not being targeted to the site of action and it is currently believed that improved efficacy in the treatment of acne may be obtained when the vitamin D analogue is targeted to the hair follicles.

Accordingly, the present invention further relates to a method of targeting a vitamin D derivative such as calcipotriol to the sebaceous glands of hair follicles, the method comprising applying on a skin area of a patient in need of such treatment, a therapeutically effective amount of a lipid nanoparticle composition as described above. More specifically, the skin area in need of treatment comprises a comedone, pustule, papule or cyst associated with acne or a related disease such as rosacea. The vitamin D derivative is preferably calcipotriol or calcipotriol monohydrate.

It is furthermore envisaged to prepare lipid nanoparticle compositions comprising both a corticosteroid and a vitamin D derivative or analogue as the active ingredients in a pharmaceutically acceptable aqueous vehicle. In this embodiment, the composition may be stabilized by addition of an antioxidant which may be selected from the group of BHA and BHT, or a mixture of BHA and BHT.

### EXAMPLES

### Example 1

### Compositions of the Invention

Solid Lipid Nanoparticles Containing Betamethasone-17-Valerate (BMV)

| | Compo- | Compo- | Compo- | Compo- | Compo- | Compo- |
|---|---|---|---|---|---|---|
| | sition A | sition B | sition C | sition D | sition E | sition F |
| Ingredient | (mg/g) | (mg/g) | (mg/g) | (mg/g) | (mg/g) | (mg/g) |
| BMV | 1 | 1 | 1 | 0.12 | 0.12 | 1.2 |
| Glycerol | 100 | — | — | 100 | 25 | 100 |
| distearate | | | | | | |
| Cetylpalmitate | — | 100 | — | — | — | — |
| Glycerol | — | — | 100 | — | — | — |
| tripalmitate | | | | | | |
| Polysorbate | 25 | 25 | 25 | 25 | 4 | 25 |
| 80 | | | | | | |
| Water | Ad 1 g | Ad 1 g | Ad 1 g | Ad 1 g | Ad 1 g | Ad 1 g |

Solid Lipid Nanoparticles Containing Betamethasone-Dipropionate (BDP)

Composition G
Excipient (mg/g)

| Unit | mg |
|---|---|
| Betamethasone-dipropionate | 0.643 |
| Glycerol distearate | 100 |
| Polysorbate 80 | 20 |
| Disodium phosphate dihydrate | 10 |
| Sodium hydroxide (hydrogenchloride) | q.s., pH 6.0 |
| Water, purified | ad 1 g |

Lipid Nanoparticles Containing Calcipotriol Monohydrate

| | Composition H | mg/g |
|---|---|---|
| 1 | Calcipotriol monohydrate | 0.0522 |
| 2 | Cetylpalmitate | 160 |
| 3 | MCT | 40 |
| 4 | Poloxamer 407 | 48 |
| 6 | Diazolidinyl urea | 5 |
| 7 | Disodium phosphate | 20 |
| | dihydrate | |
| 8 | BHA-BHT (50:50) | 3 |
| 9 | Sodium hydroxide | q.s., pH/ |
| | (hydrogenchloride) | 8.0/8.5 |
| 10 | Water, purified | ad 1 g |

| | Composition I | Mg/g |
|---|---|---|
| 1 | Calcipotriol monohydrate | 0.0522 |
| 2 | Cetylpalmitate | 200 |
| 3 | Poloxamer 407 | 48 |
| 4 | Diazolidinyl urea | 10 |
| 5 | Disodium phosphate | 20 |
| | dihydrate | |
| 6 | BHA-BHT (50:50) | 3 |
| 7 | Sodium hydroxide | q.s., pH 8.5 |
| | (hydrogenchloride) | |
| 8 | Water, purified | ad 1 g |

| | Composition J | mg/g |
|---|---|---|
| 1 | Calcipotriol monohydrate | 0.0522 |
| 2 | Cetylpalmitate | 160 |
| 3 | MCT | 40 |
| 4 | Carbomer 974 P | 10 |
| 5 | Poloxamer 407 | 48 |
| 6 | Diazolidinyl urea | 5 |
| 7 | Disodium phosphate | 20 |
| | dihydrate | |
| 8 | BHA-BHT (50:50) | 3 |
| 9 | Sodium hydroxide | q.s., pH/ |
| | (hydrogenchloride) | 8.0/8.5 |
| 10 | Water, purified | ad 1 g |

| | Composition K | mg/g |
|---|---|---|
| 1 | Calcipotriol monohydrate | 0.0522 |
| 2 | Cetylpalmitate | 200 |
| 3 | Poloxamer 407 | 48 |
| 5 | Carbomer 974P | 10 |
| 5 | Diazolidinyl urea | 10 |
| 6 | Disodium phosphate | 20 |
| | dihydrate | |
| 7 | BHA-BHT (50:50) | 3 |
| 8 | Sodium hydroxide | q.s., pH |
| | (hydrogenchloride) | 8.0/8.5 |
| 9 | Water, purified | ad 1 g |

| | Composition L | mg/g |
|---|---|---|
| 1 | Calcipotriol monohydrate | 0.0522 |
| 2 | Glycerol tristearate | 80 |
| 3 | Castor oil | 20 |
| 4 | Poloxamer 407 | 24 |
| 5 | Carbomer 974P | 10 |
| 6 | Diazolidinyl urea | 5 |
| 7 | Disodium phosphate | 20 |
| | dihydrate | |
| 8 | BHA-BHT (50:50) | 3 |
| 9 | Sodium hydroxide | q.s., pH 8.5 |
| | (hydrogenchloride) | |
| 10 | Water, purified | ad 1 g |

| | Composition M | mg/g |
|---|---|---|
| 1 | Calcipotriol | 0.0522 |
| 2 | Dynasan P60 | 80 |
| 3 | Miglyol 812 or fractionated | 20 |
| | coconut oil | |
| 4 | Poloxamer 407 | 24 |
| 5 | Carbomer 974P | 10 |
| 6 | Diazolidinyl urea | 5 |
| 7 | Disodium phosphate | 20 |
| | dihydrate | |
| 8 | BHA-BHT (50:50) | 3 |
| 9 | Sodium hydroxide | q.s., pH 8.5 |
| | (hydrogenchloride) | |
| 10 | Water, purified | ad 1 g |

| | Composition N | mg/g |
|---|---|---|
| 1 | Calcipotriol monohydrate | 0.0522 |
| 2 | Behenol | 80 |
| 4 | Poloxamer 407 | 24 |
| 5 | Carbomer 974P | 10 |
| 6 | Diazolidinyl urea | 5 |
| 7 | Disodium phosphate | 20 |
| | dihydrate | |
| 8 | BHA-BHT (50:50) | 3 |
| 9 | Sodium hydroxide | q.s., pH 8.5 |
| | (hydrogenchloride) | |
| 10 | Water, purified | ad 1 g |

Lipid Nanoparticles Containing Calcipotriol Monohydrate and Betamethasone Dipropionate

| | Composition O | mg/g |
|---|---|---|
| 1. | Calcipotriol | 0.0522 |
| 2. | Betamethasone | 0.643 |
| | dipropionate | |
| 3. | Dynasan 118 | 80 |
| 4. | Miglyol 812 | 20 |
| 5. | Poloxamer 188 | 20 |
| 6. | Carbomers 974P | 5 |
| 7. | Diazolidinyl urea | 5 |
| 8. | Disodium phosphate | 14 |
| | dihydrate | |
| 9. | BHA-BHT (50:50) | 3 |
| 10 | Sodium hydroxide | q.s., pH 6.6 |
| 11 | Water, purified | ad 1 g |

| Composition P | mg/g |
|---|---|
| Calcipotriol | 0.0522 |
| Betamethasone | 0.643 |
| dipropionate | |
| Tristearate | 100 |
| Poloxamer 188 | 10 |
| Carbomer 974P | 5 |
| Diazolidinyl urea | 5 |
| Disodium phosphate | 14 |
| dihydrate | |
| BHA-BHT (50:50) | 3 |
| Sodium hydroxide | q.s., pH 6.6 |
| Water, purified | ad 1 g |

| Composition Q | mg/g |
|---|---|
| Calcipotriol | 0.0522 |
| Betamethasone | 0.643 |
| dipropionate | |
| Dynasan 118 | 80 |
| Castor oil | 20 |
| Poloxamer 188 | 20 |
| Carbomer 974P | 5 |
| Diazolidinyl urea | 5 |
| Disodium phosphate | 14 |
| dihydrate | |
| BHA-BHT (50:50) | 3 |
| Sodium hydroxide | q.s., pH 6.6 |
| Water, purified | ad 1 g |

Calcipotriol incorporated in the lipid nanoparticles (composition H-L) was determined to be chemically stable (>90%) after 3 months of storage at 40° C.

Calcipotriol incorporated in the lipid nanoparticles (composition H-M) was determined to be chemically stable (>90%) after 18 months of storage at 25° C.

Betamethasone dipropionate was determined to be chemically stable (>90%) in Composition G after 6 months of storage at 25° C.

The stability evaluation of composition N at 25° C. is ongoing.

Calicpotriol and Betamethasone dipropionate incorporated in the lipid nanoparticles (composition O-Q) was determined to be chemically stable (>90%) in the SLN after 6 months of storage at 25° C.

Preparation of the Formulations

The preparation of SLN was done by hot high pressure homogenization as described in EP 605497.

Briefly for SLN with BMV and BDP, the lipid was melted at 80° C. and BMV or BDP was dissolved in the molten lipid during stirring. An aqueous polysorbate 80 solution of the same temperature was added to the lipid mixture and the mixture was mildly homogenized for 2 min at 6000 rpm using a Silverson High Speed Mixer L4RT from Silverson Machines Ltd. (Chesham, United Kingdom), to create a coarse emulsion. The emulsion was high pressure homogenized using an EmulsiFlex C5 from Avestin Inc. (Ottawa, ON, Canada), with the homogenizer placed in a Julabo TW 20 water bath (Seelbach, Germany) to keep the temperature at 80° C. The coarse emulsion was processed at 600 bar applying four homogenization cycles. These processing parameters were selected from preliminary studies as they resulted in SLN with a small mean particle size 200 nm) and a low polydispersity. The SLN dispersions were cooled at room temperature while protected from light and subsequently adjusted to pH 5.0. The ointment with BMV was composed of 99% (w/w) white soft paraffin and 1% (w/w) liquid paraffin and was prepared by suspending the BMV crystals in the ointment using a mortar. All formulations with BMV were stored at 5° C. protected from light until use.

The preparation of solid lipid nanoparticles and nanostructured lipid carriers comprising calcipotriol was carried out in a similar manner. Calcipotriol was dissolved in the hot oil (in case of NLC) and the mixture was added to the melted lipid. For SLN, calcipotriol was dissolved in the melted lipid and the temperature was kept at 80° C. while stirring. The poloxamer/water solution was adjusted to a pH of 8.0, heated to a temperature of 80° C. and added to the melted lipid-drug mixture. Pre-emulsions were prepared using a Silverson High Speed Mixer L4RT from Silverson Machines Ltd. (Chesham, United Kingdom). Subsequently SLN dispersions were prepared by high pressure homogenisation applying 500-800 bar and 3 cycles using an EmulsiFlex C5 from Avestin Inc. (Ottawa, ON, Canada), with the homogenizer placed in a Julabo TW 20 water bath (Seelbach, Germany) to keep the temperature at 80° C. Upon cooling, SLN was mixed with buffer, preservative and stabilizer and pH was adjusted to 8.0 or 8.5. When the formulations were thickened with carbomer it was done upon cooling by adding carbomer stepwise using a Silverson High Speed Mixer L4RT from Silverson Machines Ltd. (Chesham, United Kingdom) and then pH was adjusted as the final step.

Characterization of the Formulations

Characterization was done 0-3 days after the day of preparation, and in vivo studies were initiated within one week after the preparation.

Dynamic Light Scattering (DLS)

Particle size analysis of SLN was performed by DLS on a Zetasizer Nano ZS (Malvern Instruments Ltd., Worcestershire, UK) equipped with a 633 nm laser and 173° detection optics. Measurements were performed at 25° C. on samples (n=3) adequately diluted with purified water before measurement, i.e. the viscosity of water was used for the measurement. Malvern DTS v5.10 software was applied for the data acquisition and analysis. The particle size distribution was described by the polydispersity index (PdI) and the mean hydrodynamic diameter (Z-average).

Microscopy

The solubility of BMV in the lipid as well as appearance of the final formulations, both the SLN and the ointment, was evaluated microscopically employing a Nikon Eclipse 80i microscope equipped with a Linkam PE94 heater, both from DFA Instruments (Glostrup, Denmark). The software used for acquisition was Image Pro Plus®.

High Pressure Liquid Chromatography (HPLC)

The SLN and the ointment were analyzed quantitatively for the content of BMV, BDP and calcipotriol, respectively by HPLC.

Characteristics of SLN and Ointment

DLS analysis showed that the mean diameter of the four different SLN (only varying in the content of BMV) tested in the dose finding study was 179.9-193.6 nm with a PdI of 0.179-0.216. An optimization of the surfactant and the lipid concentrations to reduce the skin irritation while maintaining a small mean particle size and a low PdI showed that this was possible with a composition of 2.5% (w/w) distearate and 0.4% (w/w) polysorbate 80. The Z-average of the SLN with this composition was 205.4 nm and the PdI was 0.228. Both the Z-average and PdI values were dependent on the concentration of the surfactant and the lipid. For both concentrations of lipid, increasing the concentration of surfactant decreased the Z-average and PdI until a certain level of surfactant, after which the particle size did not decrease any further.

BMV was dissolved in the lipid in SLN and no drug crystals were detected by microscopy in the final formulations. In the ointment, BMV crystals (<25 μm) were distributed homogenously throughout the vehicle. The HPLC analysis showed that the concentration of BMV in the SLN and in the ointment was at the expected values with relative standard deviations of less than 1.4% (except for the 0.003 mg/g SLN in which the relative deviation was 10.3%). This illustrates that BMV was homogenously distributed and chemically stable in both formulation types.

### Example 2

### Skin Penetration Studies

Skin Sample Preparation

The porcine ears were obtained from newly slaughtered pigs from the Danish Meat Trade College (Roskilde, Denmark). The ears were stored at −20° C. and thawed slowly at 4° C. before shaving and removing full-thickness skin from the back of the ears using a scalpel. Subcutaneous tissue was carefully removed with a scalpel and the skin was cut into appropriate pieces before freezing at −20° C. until use (after no more than 14 days). Two skin pieces were obtained from each ear, and they were balanced with respect to intact and barrier-impaired skin.

Skin barrier impairment was induced by 25 successive tape strippings applying D-Squame® tape discs (Cuderm Corp., Dallas, USA). 225 g/cm² pressure on the tape was applied with a D-Squame® tape applicator for 5 sec. (Cuderm Corp., Dallas, USA). The skin was mounted on a cork plate with small pins, stretching it to overcome problems with skin furrows when tape stripping. The method was adapted from Simonsen et al. who created a skin model to simulate barrier properties of AD skin [L. Simonsen and A Fullerton, Skin Pharmacol. Physiol. 20(5), 2007, pp. 230-236]. The effect of tape stripping fresh versus frozen and thawed skin was validated by an initial study comparing fresh and thawed skin (n=6) and tape stripping 0, 5, 15, 25 and 40 times. Punch biopsies were taken and fixed in 10% (w/w) formalin followed by haematoxylin-eosin staining before examination by microscopy.

Skin Penetration and Permeation Studies

Penetration profiles of BMV and lipid were evaluated. ³H-labelled BMV and ¹⁴C-labelled lipid were used to study the penetration of BMV and lipid particles (distearate and tripalmitate) into intact and barrier-impaired skin.

The skin was mounted on Franz type diffusion cells with the dermis side facing the receptor medium (diffusion area 3.14 cm², recipient volume 10 ml, constant stirring, temperature kept at 32° C.). A solution of 1% (w/w) methyl-β-cyclodextrin in isotonic acetate buffer pH 5.5 (15 mM sodium acetate, 100 mM sodium chloride) was used as receptor medium. After equilibrating the skin for 30 min with isotonic sodium chloride solution and for 60 min with receptor medium, the receptor medium was renewed and 20 mg formulation (6.4 mg formulation/cm²) applied evenly on the skin surface using a spatula. The exact amount of formulation applied was determined by weighing the spatula before and after application. After complete incubation, the skin was separated and analyzed. All studies were done on intact or barrier-impaired skin and the formulation application time was varied between 6, 16 and 24 h. Occlusion was created by mounting a glass plug on the top of the skin to ensure complete occlusive conditions. After complete application time surplus formulation was removed by wiping the skin twice with a cotton pad. 10 ml of ethylacetate was used to extract BMV and lipid from the cotton pads and from the lid of the donor compartment. Heptane:ethanol (30:70) was used to extract BMV from the ointment. The stratum corneum was removed by applying a maximum of 15 tape strips to intact skin using D-Squame® tape discs (Cuderm Corp., Dallas, USA) applying the same technique as when inducing skin damage. For barrier impaired skin only a maximum of 3 tape strippings was done. In both cases, the first tape strip was included as surplus formulation. The method for removing stratum corneum was established by preliminary studies with different formulations and tests results using different numbers of tape strips after 24 h of exposure to the formulation. If however the epidermis started to loosen with less than 15 or 3 tape strips, respectively, tape stripping was ended and the last strip included in the epidermis count. Epidermis and dermis were separated by heat (incubation at 5 min at 60° C. and high humidity). The skin surrounding the application area (designated non-applied skin) was cut into small pieces and analyzed as well to include any lateral penetration and to achieve full recovery. Soluene® 350 was added to the tape strips and to the skin samples to solubilize the tissue and extract drug substance and lipid. After 24 h of incubation at 50° C., 10 ml of Hionic-Fluor was added to the Soluene® 350 samples and analyzed by liquid scintillation in a Tri-Carb 2100 TR Liquid Scintillation Analyzer from Packard Instrument Company (Meriden, USA). The extractions from the lid and the cotton pad and the content in the receptor medium were analyzed by mixing 1 ml with 10 ml of Hionic-Fluor before scintillation counting. The appropriate liquid (i.e. ethylacetate, receptormedium, heptane:ethanol (30:70) and Soluene® 350 mixed with Hionic-Fluor) was used as background measurements.

When comparing 6, 16 and 24 h the amount of BMV in the different skin layers were pooled to take into account the fact that the skin structure changes during 24 h, so that the efficiency of the separation procedure may also change.

In vitro penetration studies were done on intact and barrier-impaired porcine skin in Franz diffusion cells varying the SLN lipid component, duration of application and the presence of occlusive conditions in order to evaluate how SLN affect the drug substance penetration profile into and across the skin.

The ratio of BMV to distearate was found to increase with skin depth, i.e. more BMV than lipid penetrated the skin. The ratio in the surplus formulation was approximately 1. In stratum corneum, it decreased to below 1, and subsequently increased through epidermis and dermis to reach a ratio of 3.6 in the receptor medium. Compared to distearate, the tripalmitate profile was slightly different, as a decrease in the BMV to lipid ratio was seen in the dermis, and the ratio in the receptor medium was 2.7, which was lower than for distearate. The fact that the BMV/lipid ratio increased with skin depth indicates that the lipid particles stayed mainly on the surface or in the upper layers of the skin, and that BMV was released from the particles to penetrate into the skin. Table 3 shows the penetration data of lipid into the different skin layers and into the receptor medium. There was a tendency that more distearate than tripalmitate reached the epidermis, both for intact and barrier-impaired skin. When the skin was barrier impaired by tape stripping, an increase in the amount of lipid reaching the epidermis, dermis and receptor medium was seen. The increase in lipid penetration (˜3-7 fold) was very low compared to the increase in BMV penetration (˜3-60 fold) in barrier-impaired skin, emphasizing that BMV was released from the nanoparticles and diffused separately through the skin.

The main difference observed between the SLN and the ointment after skin application for 24 h was that SLN caused a significantly higher total amount of BMV to remain in the skin both in intact and barrier-impaired skin (FIG. 1). When the barrier was intact, a large amount of BMV administered in SLN was found in the stratum corneum and less in the receptor medium (FIG. 1a). SLN caused BMV to localize to a higher degree to the upper layers of the skin from where it could diffuse into deeper skin layers and it was apparent that SLN caused significantly more BMV to penetrate deeper into the stratum corneum of intact skin when compared to the ointment (p<0.05). With cetylpalmitate SLN more BMV reached the epidermis. Only a negligible amount of BMV (<0.9%) permeated to the receptor medium when SLN were applied to the intact skin whereas significantly more (8.4±4.0%) permeated the intact skin after using the ointment (FIG. 1a). Upon tape stripping the skin prior to application of either SLN or ointment, permeation of the BMV from all formulations into the receptor medium was greatly enhanced (13.8-21.8%) (FIG. 3b). Significantly more BMV remained in the barrier-impaired skin upon application of SLN as compared to the ointment (p<0.05), but a similar amount of BMV permeated to the receptor medium (FIG. 1b). The tendency was that more BMV was retained in the stratum corneum both in intact and barrier-impaired skin after administration of BMV in distearate SLN as compared to tripalmitate and cetylpalmitate SLN (FIG. 1).

To achieve a better understanding of the results obtained after 24 h of application two additional time points were included in the study, viz. 6 and 16 h. Distearate was chosen for these studies due to the higher solubility of BMV in this lipid. After application for 6, 16, and 24 h respectively, it was shown that at all time points significantly lower amounts of BMV was present in the skin when using the ointment (FIG. 2) and that application of distearate SLN resulted in a constant and significantly higher amount of BMV in the skin. The amount of BMV permeating to the receptor medium for barrier-impaired skin was increased only to a small extent in 24 h when administered in SLN (FIG. 2b).

When the skin was immediately occluded for 24 h after application of SLN, the amount of BMV that permeated to the receptor medium was significantly increased as compared to non-occluded conditions and the amount in the skin was lowered (FIG. 3). Upon occlusion and in contrast to the intact skin, the increase in BMV that permeated to the receptor medium was extremely high for barrier-impaired skin; 65.6±15.2% when distearate SLN was applied (FIG. 3b).

In Vivo Dermal Delivery of Betamethasone Dipropionate in Hairless Rats

Under isoflurane anaesthesia, 100 μl of the formulation was applied to a 4×3 cm area at the back of the rat. The rat was left for 2 minutes (for the formulation to dry) and a “biofilm”, Optiskin film (5.3×7.2 cm, URGO laboratories, France) was applied over the area and on top of that, Fixomull strech (BSN medical, Germany).

The rats were terminated at different time intervals post dose at which point skin biopsies and blood samples were taken. Sublingual blood samples were collected from each animal prior to termination. The rats were euthanized with CO₂. Skin biopsies were taken from the applied skin area. The skin was gently cleaned with a tissue soaked in 99.9% ethanol. The biopsies were weighed and kept at −80 deg until quantitative analysis. Quantitative analysis was carried out by LC-MS/MS.

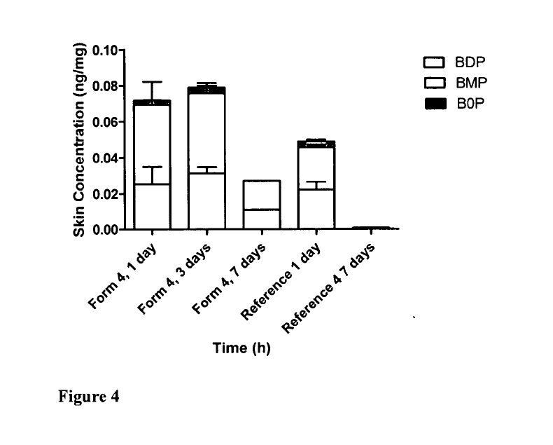

FIG. 4 illustrates that a higher level of betamethasone dipropionate (BDP) and its metabolites betamethasone (BOP) and betamethasone-monopropionate (BMP) may be obtained in the skin for a longer period of time by administration in SLN to the skin of hairless rats when compared to an ointment. On day 3 a steady high level of the drug substance in the skin was achieved and on day 7 the drug substance still remained in the skin to some extent while it could not be detected when administered in an ointment. These data confirm the reservoir effect seen in vitro when the corticosteroid BMV in SLN was applied to intact and barrier-impaired porcine skin.

### Example 3

### Evaluation of Lipid Nanoparticles Containing BMV as a Cutaneous Delivery System in a Murine Atopic Dermatitis Model

AD Induction

The AD phenotype was induced by repeated challenges with oxazolone (Man et al., J. Invest. Dermatol. 128, 2008, pp. 79-86). The mice were sensitized by application of 10 μl 0.8% (w/v) oxazolone in acetone to each side of the right ear (designated day −7). A control group was treated with 10 μl of acetone on both sides of the right and left ear. Seven days after the sensitization (designated day 0), the mice were challenged for the first time with 0.4% (w/v) oxazolone in acetone. More specifically, the mice were dosed with 10 μl of 0.4% (w/v) oxazolone on each side of the right ear every other day from day 0 to day 21. On the same days as the oxazolone was applied, mice in the control group were dosed with 10 μl of acetone only on each side of the left and right ears, respectively. The treatment was initiated on day 10 comprising a once daily treatment scheme until the study was ended at day 21. The following read outs were measured; ear thickness, TEWL, cytokine analysis, histological assessment of the ear skin and quantitative analysis of BMV in ear tissue and serum.

Dose Finding for BMV Administered in SLN

An initial study was carried out in order to find the dose response relationship for BMV in the SLN and to establish a dose to be used when comparing the effect of the SLN with an ointment. Three different concentrations of BMV in the SLN were applied in this study. The mice were divided into 8 groups of 4-8 animals (total of 52 mice) comprising two acetone control groups of mice (n=4) with no AD induced and which were treated once daily with either 10 μl of acetone on each side of the right and the left ear or once daily with 5 μl of placebo SLN on each side of the right ear. The other groups consisted of mice that had AD induced by oxazolone and that were treated once daily on each side of the right ear with either 10 μl of acetone (n=7), 10 μl of 3.0 μg/ear/day BMV in acetone (n=6), 5 μl of placebo SLN (n=7), 5 μl of 0.030 μg/ear/day BMV in SLN (n=8), 5 μl of 1.20 μg/ear/day (n=8) and 5 μl of 12.0 μg/ear/day BMV in SLN (n=8). The left ear was untreated

Irritation and Effect of SLN and an Ointment

The composition of the SLN was optimized in a study in which the irritative effect of the SLN vehicle on the mouse ear skin was evaluated. Specifically, the concentration of the surfactant polysorbate 80 was evaluated. A range of placebo formulations were produced to find the optimum levels of surfactant and lipid that would result in a mean particle size of approximately 200 nm and a low particle polydispersity (<0.25). Moreover, particles of this composition should not induce any irritative effects on the healthy mouse ear skin and should provide sufficient BMV solubility in the lipid mixture (>0.012% (w/w)). The composition of a SLN vehicle that met these criteria was found to be 2.5% (w/w) lipid and 0.4% (w/w) polysorbate 80 (Table 1). The effect of this SLN formulation was compared to a conventional paraffin ointment, which was also shown to be non-irritative to the healthy mouse ear skin.

To compare SLN with an ointment for delivery of BMV, 69 mice were divided into 8 groups of 4-10 animals of which one group was an acetone control group (n=4) with no AD induced. Mice in the other groups all had AD induced by oxazolone and they were treated once daily on each side of the right ear with; 10 μl of acetone (n=8), 10 μl of 3.0 μg/ear/day BMV in acetone (n=8), 10 μl of 1.20 μg/ear/day BMV in acetone (n=10), 5 μl of 1.20 μg/ear/day BMV in SLN (n=10), 6 μg of 1.20 μg/ear/day BMV in ointment (n=10), 5 μl of placebo SLN (n=10) and 6 μg of placebo ointment (n=9). Treatment scheme was otherwise similar to the dose finding study. SLN were applied with a pipette and a spatula was used for the ointment due to the higher viscosity. The compositions of the formulations tested are shown in Table 1.

Ear Thickness

As a measure of the degree of skin inflammation, the ear thickness of the right and the left ears was measured using a digital micrometer from Mitutoyo Americ Co. (Aurora, Ill., USA), using the ear thickness value of the untreated ears as an internal control. All measurements done throughout the studies were carried out by the same person in order to minimize interindividual measurement variability. The measurements were performed before the treatment was applied on day 10, 12, 14, 17, 19 and 21.

Transepidermal Water Loss (TEWL)

TEWL was used as an indicator of the skin barrier condition applying a Vapometer from Delfin Technologies Ltd. (Kuopio, Finland). TEWL was measured in 20 s on the right ear of the mouse (once per mouse), using a nail adaptor with a diameter of 4.5 mm. The measurements were carried out before the treatment was applied on day −7, 10, 14 and 20 in the dose-finding study and on day −7, 10, 13, 17 and 20 in the study where SLN was compared to an ointment. Ambient relative humidity and temperature in the laboratory was measured upon each TEWL measurement.

Endpoint Sampling

On day 21, two hours after the last application of the formulations, blood and the right ear were sampled from all animals. In the acetone control groups, both ears were sampled. The animals were anaesthetized with isoflurane and blood was sampled from the eye using a capillary tube. The blood was filled into 2.5 ml Vacutainer® vials and left to stand for 30 min at room temperature followed by centrifugation for 10 min at 1000 G and 4° C. The supernatant was transferred to a 1.4 ml non-coded u-bottom bulk Micronics tube and stored at −80° C. until quantitative drug analysis. After blood sampling, the animals were sacrificed and the right ear was cut of with a scalpel Using a Stiefel® biopsy punch (Offenbach am Main, Germany) an 8 mm biopsy from the middle of the ear was isolated for tissue analysis and from here a 3 mm biopsy was taken and preserved in 10% (v/v) neutrally buffered formaldehyde. The remaining ear tissue from the 8 mm biopsy was cut into two halves, one of which was tape stripped before analysing the drug concentration and the other was used for cytokine analysis. Both were snap-frozen in liquid nitrogen and stored at −80° C. until the analysis was carried out.

Cytokine Level in Ear Tissue

The ear tissue was homogenized in 200 μl lysis buffer using a Precellys® 24 tissue homogenizer with a Cryolys cooling unit from Bertin Technologies (Montigny-le-Bretonneux, France). The lysis buffer was composed of 1 mM Na₃VO₄, 0.4% (v/v) nonyl phenoxypolyethoxylethanol (NP40) and the protease inhibitor Complete™ from Roche Diagnostics (Mannheim, Germany) dissolved in PBS. After homogenization, the samples were left on ice for a period of 15-30 minutes before centrifuging at 4° C. and 15000 G for 15 minutes using a Microcentrifuge 157 MP from Ole Dich Instrument makers (Hvidovre, Denmark). The supernatant was kept at −80° C. until the MSD® cytokine assay was performed. A determination of the total protein concentration was performed to normalize the sample concentration before the cytokine determination. It was carried out using a Pierce® BCA Protein Assay kit from Pierce Biotechnology (Rockford, Ill., USA). Plates were read on a VICTOR™ X3 Multilabel Plate Reader from Perkin Elmer (Skovlunde, Danmark). Specific level of the cytokines IFN-γ, IL-1-β, IL-2, IL-4, IL-5, IL-8, IL-10, IL-12 total and TNF-α in the ear tissue were determined with a MSD® mouse TH1/TH2 9-plex Multi-Spot® 96-well 10 spot plate using a MSD® Sector Imager 6000 (Meso Scale Discovery, Gaithersburg, Md., USA). The MSD® assay was optimized for tissue use. Thus, the plate was blocked for 1 hour with 150 μl MSD calibrator blend, followed by 3 times washing with 150 μl 0.05% (v/v) polysorbate 20. 25 μl diluted tissue supernatant samples were added to the plate followed by shaking for 2 hours at room temperature. Then, 25 μl SULFO-TAG™ detection antibody solution (Meso Scale Discovery, Gaithersburg, Md., USA) was added and incubated for 2 hours at room temperature. Washing was repeated 3 times with 0.05% (v/v) polysorbate 20 before finally adding 150 μl read buffer diluted 1:2 in MilliQ water and reading the plate on the MSD® Sector Imager 6000.

Quantitative Analysis of BMV in Ear Tissue and Serum

Quantitative analysis of BMV in serum and tissue was carried out using mass spectrometry (MS). 300 μl 5 mg/ml proteinase K solution and 1700 μl digestion buffer pH 8.5 (1.58% trizma hydrochloride, 0.029% EDTA, 0.20% sodium dodecyl sulphate, 0.117% sodium chloride), was added to the ear tissue sample followed by sonication for 20 min to homogenize the sample. 50 μL of the sample was transferred to a deepwell plate and precipitation was induced by addition of 150 μL methanol containing the internal standard EO1271 (20 nM). After centrifugation at 4000 rpm for 30 min at 10° C. the samples were analyzed. To precipitate the protein in the serum samples 100 μl internal standard in acetonitrile was added. 20 μl sample and standard, respectively, was transferred to a deepwell plate. After gentle shaking the plate was centrifuged at 4000 rpm for 30 min at 10° C. The samples were analysed on a LC-MS/MS system with a UPLC from Waters (Milsford Mass., USA) and an API5000 Mass Spectrometer from Applied Biosystems (Carlsbad, Calif., USA). Analyst (API5000) and Waters Acquity software was applied for the data acquisition and analysis. The temperature was 60° C. and the flow was 0.5 ml/min. A flow gradient method was used varying the mobile phase from 0-100% of methanol:1 M ammoniumacetate:formic acid:water (900:2:0.755:100) and of methanol:1 M ammoniumacetate:formic acid:water (50:2:0.755:950) during 2.3 min. The detection limit was determined to be 11.11 ng/mg for the skin samples, and 0.001 ng/ml for the serum samples.

Data Analysis

All data were plotted in Microsoft Excel or Graph Pad Prism 5.0. Statistical analysis was performed in Graph Pad Prism 5.0. One way ANOVA (p<0.05) followed by Newman Keuls or Dunnets multiple comparison test to compare means was done.

AD Model Validation

The chronic oxazolone model is a well validated and accepted model for AD (Man et al., supra. Indeed, our animal model showed to have a well induced AD phenotype too. The ear thickness of ears treated with oxazolone was increased while no change in the ear thickness was seen for the ears treated with acetone only. As expected, treatment with BMV in acetone caused a significant reduction in ear thickness in the oxazolone sensitized mice. Moreover, the TEWL values correlated to the ear thickness data confirming skin inflammation and barrier impairment. Thus, the TEWL values increased from 10 g/m²*h to 30-35 g/m²*h upon 10 days of oxazolone treatment. The cytokine expression was up-regulated in the group sensitized with oxazolone while it was not affected in the mice treated with acetone only. The cytokine level was down-regulated in the oxazolone sensitized mice upon treatment with BMV in acetone, confirming the efficacy of topical corticosteroid treatment. Histological assessment also clearly showed marked inflammation responses in ear tissue of the oxazolone sensitized mice, on the contrary skin samples from the mice treated with acetone only, showed no signs of inflammation while skin samples from mice treated with BMV showed only mild signs of inflammation correlating with the efficacy of the drug treatment (data not shown).

Similar Ear Thickness Reduction of BMV in SLN and an Ointment

Ear thickness was chosen as one of the end points as it can be used as a measure of the degree of skin inflammation (Patrick et al., Toxicol. Appl. Pharmacol. 81, 1985, pp. 476-490; Young et al., J. Invest. Dermatol. 82, 1984, pp. 367-371).

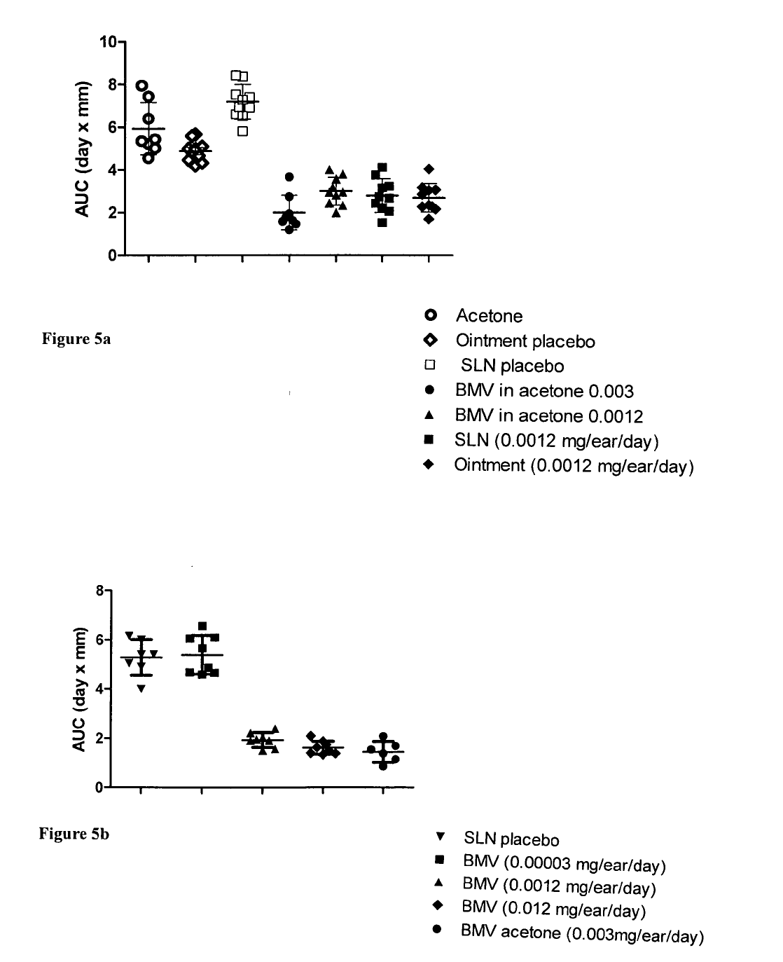

The results of the ear thickness measurements showed that SLN and an ointment with BMV caused a similar and significant (P<0.05) reduction in the ear thickness and that the effect was in the same range as the 1.20 μg/ear/day BMV in acetone control (FIG. 5a). This strongly indicates that BMV was released from the SLN vehicle and penetrated the skin to reach the target cells in an amount high enough to exert an effect comparable to the ointment and to BMV administered in acetone. Previously, it was shown in vitro that when BMV in SLN was applied to porcine skin, the total amount of drug substance that remained in the skin, and distinctively in the stratum corneum, was significantly higher than when BMV was applied in an ointment, cf. Example 2. However, it is imperative that the drug substance is released from the SLN and penetrates the stratum corneum in order to exert an effect, which is confirmed here in vivo.

In the dose-finding study there was a distinct dose response effect on the ear thickness reflected as a significant (P<0.05) decrease in the ear thickness after application of SLN with BMV in the concentrations 1.20 μg/ear/day and 12.0 μg/ear/day when compared to the untreated animals as well as the SLN placebo group (FIG. 5b). No effect was seen from treatment with 0.03 μg/ear/day of BMV in SLN.

Cytokine Levels Demonstrate Similar Effect of BMV in SLN and an Ointment

Multiple challenges with oxazolone have been shown to cause a shift in the skin inflammation from a mainly Th1 dominated to a Th2 dominated response reflected in the expression of cytokines, which is commonly seen in human AD (Kitagaki et al., J. Invest. Dermatol. 105, 1995, pp. 749-755; Kitagaki et al., J. Immunol. 159, 1997, pp. 2484-2491; Matsumoto et al., Skin Res. Technol. 10, 2004, pp. 122-129).

Both Th1 and Th2 producing cytokines were evaluated in this study. In the dose-finding study there was a clear dose response effect of BMV in SLN, i.e. the level of IL-10, IL-4, IL-8, IL-10 and IL-12 was reduced in proportion to the dose of BMV applied in SLN (data not shown). Unfortunately, the concentration of IL-5 and TNF-α was below the detection limit upon treatment with BMV why these cannot be included in the data analysis. Compared to the acetone control, the IL-8 and IL-12 levels were significantly increased after application of placebo SLN to mice not sensitized with oxazolone indicating irritation from the SLN vehicle.

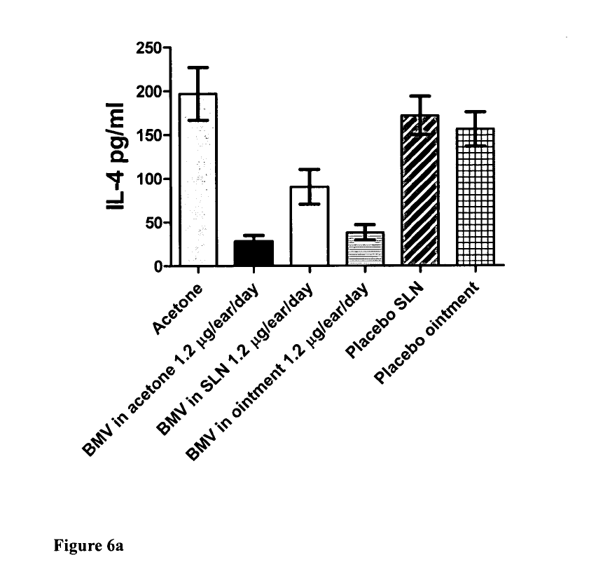

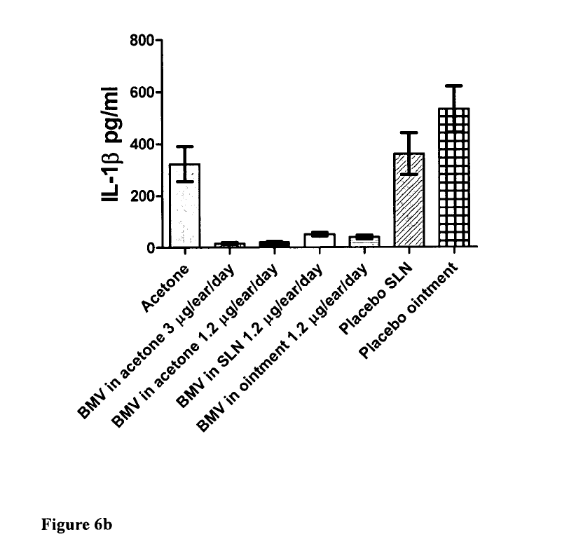

The comparison of BMV in SLN with BMV in an ointment showed that in both formulations BMV down-regulated the level of IL-1β, IL-4, IL-8, IL-10 and IL-12 as was also seen in the dose-finding study. This is exemplified in FIGS. 6a and 6b which show the downregulation of the cytokines 11-4 and IL-1β. There were no significant differences between the formulations regarding the cytokine regulation, meaning that the two formulations were equally effective in this respect. To note is though, that the cytokine levels after application of the placebo ointment were generally higher than after application of the placebo SLN, indicating that the ointment vehicle was more irritative than SLN.

TEWL is Reduced by BMV Administered in SLN or an Ointment

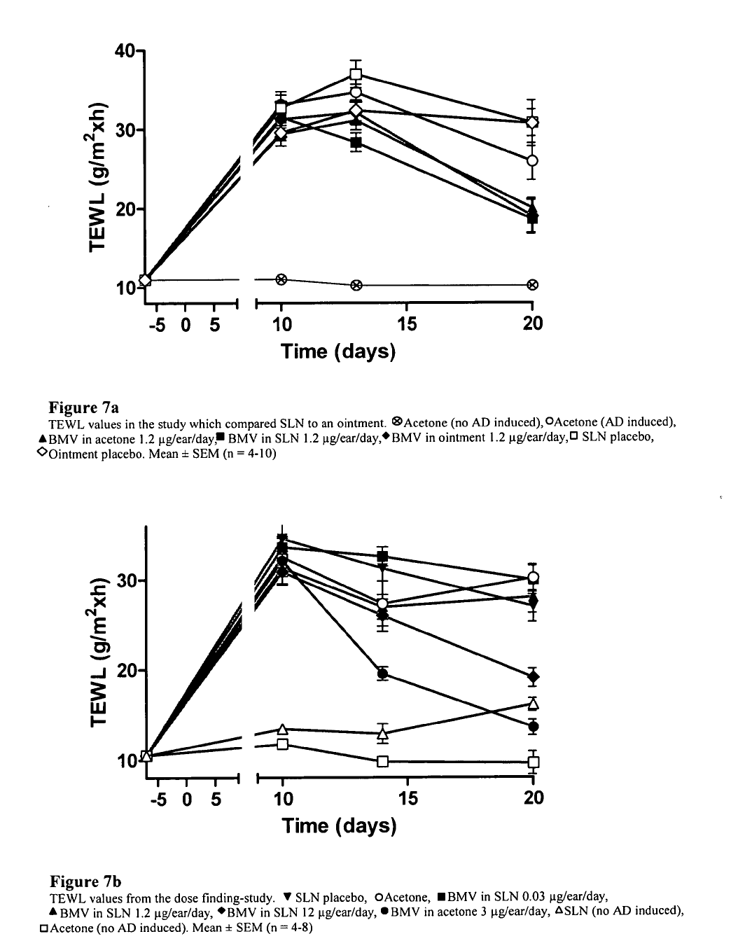

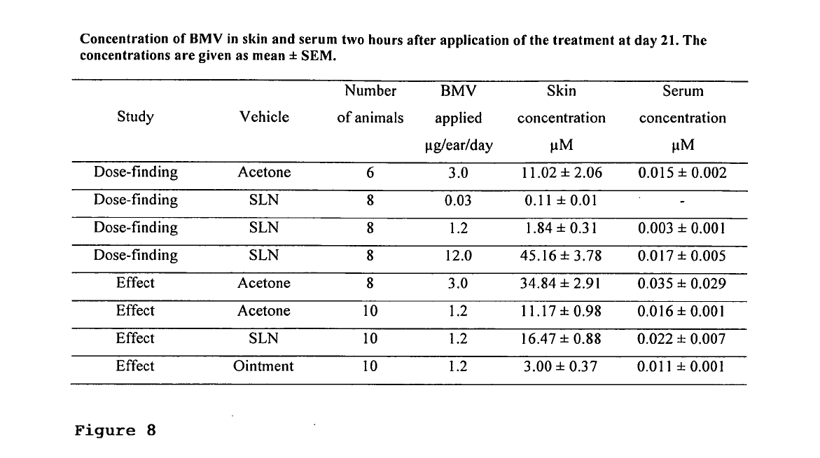

TEWL can be used as an indicator of the skin barrier properties (Levin and Maibach, J. Contr. Rel. 103, 2005, pp. 291-299; Proksch et al., J. Dermatol. Sci. 43, 2006, pp. 159-169; Werner and Lindberg, Acta Derm. Venereol. 65, 1985, pp. 102-105) and to evaluate the occlusive properties of a vehicle applied to the skin (Loden, Acta Derm. Venereol. 72, 1992, pp. 327-330). Comparison of SLN with an ointment applying 1.20 μg BMV/ear/day showed that treatment with either formulation decreased the TEWL values significantly to a level, which was comparable to the control (FIG. 7a). In the dose-finding study, only treatment with BMV in SLN 1.2 μg/ear/day caused a significant (P<0.05) reduction in the TEWL value (FIG. 7b). In contrast to previous studies performed with SLN applied to the skin (Jenning et al., Int. J. Pharm. 199, 2000, pp. 167-177; Santos et al., J. Drug Target. 10, 2002, pp. 489-495; Wissing and Muller, Eur. J. Pharm. Biopharm. 56, 2003, pp. 67-72) TEWL did not indicate any occlusive effects from placebo SLN, and neither from the placebo ointment. TEWL was decreased only by the formulations with BMV included. The reason why the occlusive effect was not apparent may be that the measurements were done the day after the formulations were applied. By this time, the formulation may not have been present on the skin in an amount high enough to induce occlusion especially as the mice scratched and cleaned their ears continuously. The decrease in TEWL after administration of the formulations with BMV was therefore thought to be related to the therapeutic effect of BMV, specifically the vasoconstrictive effect, which may reduce the evaporation of water from the skin surface (Kolbe et al., Skin Re. Technol. 7, 2001, pp. 73-77).

SLN Increases the Amount of BMV in the Skin

The concentration of BMV in skin and serum was analyzed two hours after the last application on day 21. Comparison of SLN with an ointment showed that the skin concentration of BMV after application of SLN was significantly higher (5-fold) than after application of BMV in the ointment. The concentration of BMV in the serum was similar for SLN, ointment and the acetone control 1.20 μg/ear/day (FIG. 8). The dose-finding study showed that the level of BMV in the serum was comparable after application of SLN with BMV 12.0 μg/ear/day and BMV in acetone 3.0 μg/ear/day (FIG. 8). The level in the skin, however, was higher for SLN with BMV 12 μg/ear/day than for BMV in acetone 3.0 μg/ear/day. SLN 0.03 μg/ear/day could not be measured in the serum, and a very low level of BMV was present in the skin explaining that no effect was seen with this formulation either.

The dose response seen in the ear thickness, the TEWL and the ear tissue cytokine level together with the skin concentrations achieved from the different formulations applied including the controls indicate that BMV penetrated percutaneously and exerted a local effect. However, the possibility of the mice ingesting BMV orally when they scratch and clean their ears cannot be out ruled in the interpretation of the serum concentrations. The fact that more BMV was found in the skin after application of SLN may be an indication of an ability of SLN to adhere more strongly to the skin and create a skin reservoir as compared to the ointment. This effect was also seen in vitro when SLN with BMV were compared to an ointment, cf. Example 2. In the present study, it was shown that besides causing an increased amount of drug substance to remain in any remaining stratum corneum and/or in the viable epidermis drug substance from SLN also reached the target site to an extent that created an equal effect to a conventional formulation and an acetone control. A similar result was found in another in vivo study comparing the effect of cyclosporin A applied in either SLN or an oil in a murine AD model. It was demonstrated that cyclosporin A in SLN was more effective than an oil and that the amount of drug substance in the stratum corneum of the skin was significantly increased with the use of SLN (Kim et al., Pharmazie 64, 2009, pp. 510-514). Another study, performed in eczema patients, showed that treatment with clobetasol propionate incorporated in SLN induced a skin reservoir effect and was superior in efficacy compared to a marketed cream. (Kalariya et al., Indian J. Exp. Biol., 2005, pp. 233-240)

### Example 4

Skin penetration and permeation studies were performed with the compositions H, L, J, M, N as described in example 2 with the exception that only intact pig ear skin was used and the receptor medium was replaced with 4% BSA. The data from these studies showed that the lipid nanoparticles were able to retain calcipotriol in the stratum corneum and specifically in the viable skin when compared with the cream. Furthermore, a very small amount of the drug substance permeated through the skin to the receptor medium when applying the lipid nanoparticles.

1. A pharmaceutical composition comprising, as a therapeutically active ingredient, a corticosteroid incorporated as a solid solution or dispersion in lipid nanoparticles, said lipid nanoparticles being solid at ambient temperature and comprising about 60-92% by weight of a first lipid with a melting point above body temperature, said first lipid being a wax selected from the group consisting of esters of C₁₂₋₂₄ alcohols and C₁₂₋₂₄ fatty acids, glyceryl mono-, di- or triesters of C₁₂₋₂₄ fatty acids, C₁₂₋₂₄ fatty alcohols, and cholesterol, said lipid nanoparticles further comprising about 2-25% by weight of a pharmaceutically acceptable surfactant.

2. A composition according to claim 1, wherein the first lipid comprises about 65-92% by weight, or about 70-90% by weight, or about 75-85% by weight, or about 80% by weight, of the lipid nanoparticles, and the surfactant comprises about 8-22% by weight, such as about 10-20% by weight, of the composition.

3. A composition according to claim 1, wherein the first lipid is selected from the group consisting of cetylpalmitate, beeswax, stearyl palmitate, stearyl behenate, glycerol monostearate, glycerol distearate, glycerol dibehenate, glycerol trimyristate, glycerol tripalmitate, glycerol tristearate, behenol, stearic acid, hydrogenated palm oil, hydrogenated coco-glycerides, hydrogenated castor oil, and cetostearylalcohol.

4. A composition according to claim 1, wherein the surfactant is selected from the group consisting of poloxamers, polysorbates, sugar esters, ethoxylated fatty alcohols or phospholipids.

5. A composition according to claim 1 further comprising about 1-40%, such as about 10-30% by weight or about 15-25% by weight or about 20% by weight, of the lipid nanoparticles of a second lipid which is an oil at ambient temperature and miscible with the first lipid, or a lipophilic emulsifier or emollient.

6. A composition according to claim 5, wherein the second lipid is selected from the group consisting of a C₆₋₁₀ monoglyceride, C₆₋₁₀ diglyceride, isopropyl myristate or isopropyl palmitate, medium chain triglycerides such as caprylic/capric triglycerides, or long chain triglycerides including vegetable oils such as castor oil, sunflower oil, safflower oil, evening primrose oil, borage seed oil, sesame oil, corn oil, palm kernel oil, olive oil, avocado oil, almond oil, rapeseed oil, coconut oil, cottonseed oil, peanut oil, soybean oil, wheat germ oil, grape kernel oil or jojoba oil.

7. A composition according to claim 5, wherein the first lipid comprises about 75-85% by weight of the lipid nanoparticles, the second lipid comprises about 15-25% by weight of the lipid nanoparticles, and the surfactant comprises about 2-5% by weight of the lipid nanoparticles.

8. A composition according to claim 1, wherein the corticosteroid is selected from the group consisting of amcinonide, betamethasone, budenoside, clobetasol, clobetasone, cortisone, desonide, desoxycortisone, desoximethasone, dexamethasone, diflucortolon, diflorasone, flucortisone, flumethasone, flunisolide, fluocinonide, fluocinolon, fluorometholone, fluprednisolone, flurandrenolide, fluticasone, halcinonide, halobetasol, hydrocortisone, meprednisone, methylprednisone, mometasone, paramethasone, prednicarbate, prednisone, prednisolone and triamcinolone or a pharmaceutically acceptable ester or acetonide thereof.

9. A composition according to claim 8, wherein the corticosteroid is betamethasone or an ester thereof, such as betamethasone-17-valerate or betamethasone-17,21-dipropionate.

10. A composition according to claim 1, wherein the lipid nanoparticles have a mean/average particle size/diameter in the range of about 10-800 nm, in particular about 50-600 nm, such as 100-500 nm.

11. A composition according to claim 1, wherein the lipid nanoparticles are present in an amount of about 1-40% by weight, such as about 5-30% by weight or about 10-20% by weight of the composition.

12. A composition according to claim 1 further comprising an aqueous phase.

13. A composition according to claim 12, wherein the aqueous phase comprises a thickener.

14. A composition according to claim 13, wherein the thickener is selected from the group consisting of a carbomer, a cellulose derivative such as hydroxypropylcellulose, hydroxypropylmethylcellulose, hydroxyethylcellulose, hyaluronic acid, alginate, dextran or a derivative thereof.

15. A composition according to claim 13, wherein the thickener is present in an amount of about 0.1-5% by weight, such as about 0.5% by weight, of the composition.

16. A method of preparing a composition according to claim 1, the method comprising
(a) solubilizing or dispersing a corticosteroid in an lipid phase comprising about 60-92% by weight of a first lipid with a melting point above body temperature, said first lipid being a wax selected from the group consisting of esters of C₁₂₋₂₄ alcohols and C₁₂₋₂₄ fatty acids, glyceryl di- or triesters of C₁₂₋₂₄ fatty acids, C₁₂₋₂₄ fatty alcohols, and cholesterol, said first lipid being in a molten state, said lipid phase optionally comprising about 1-40% by weight of a second lipid which is an oil at ambient temperature and miscible with the first lipid, or a lipophilic emulsifier or emollient;
(b) dispersing the lipid phase obtained in step (a) in an aqueous phase comprising 0.2-10% by weight of a pharmaceutically acceptable surfactant selected from the group consisting of a poloxamer, a polysorbate, a sugar ester or an ethoxylated fatty alcohol to form an emulsion,
(c) homogenizing the emulsion obtained in step (b) using a high-pressure homogenizer to form nanoparticles of the lipid phase comprising the corticosteroid and
(d) cooling the homogenized emulsion obtained in step (c) to solidify the nanoparticles.

17. A composition according to claim 1 for the prevention or treatment of inflammatory or hyperproliferative skin diseases such as psoriasis, eczema, atopic dermatitis, contact dermatitis, skin ageing, photoageing, acne, urticaria or pruritis.

18. A topical pharmaceutical composition comprising, as a therapeutically active ingredient, a vitamin D derivative incorporated as a solid solution or dispersion in lipid nanoparticles, said lipid nanoparticles being solid at ambient temperature and comprising about 60-92% by weight of a first lipid with a melting point above body temperature, said first lipid being a wax selected from the group consisting of esters of C₁₂₋₂₄ alcohols and C₁₂₋₂₄ fatty acids, glyceryl triesters of C₁₂₋₂₄ fatty acids with an acid value of 0.1 or less, C₁₂₋₂₄ fatty alcohols, and cholesterol, said lipid nanoparticles further comprising about 2-22% by weight of a pharmaceutically acceptable surfactant selected from the group consisting of poloxamers and ethoxylated fatty alcohols.

19. A composition according to claim 18, wherein the first lipid comprises about 65-90%, or about 70-85%, or about 75-80% by weight of the lipid nanoparticles.

20. A composition according to claim 18, wherein said first lipid is selected from the group consisting of cetylpalmitate, a C₁₄₋₂₈ fatty alcohol, hydrogenated palm oil and triglycerides with an acid value of 0.1 or less.

21. A composition according to claim 18, further comprising about 1-40% by weight, or about 10-35%, or about 15-30%, or about 20-25% by weight of the lipid nanoparticle of a second lipid which is an oil at ambient temperature and miscible with the first lipid.

22. A composition according to claim 18, wherein said second lipid is selected from the group consisting of isopropyl myristate or isopropyl, palmitate medium chain triglycerides, such as caprylic/capric triglycerides, or long-chain triglycerides, such as castor oil.

23. A composition according to claim 18, wherein the lipid nanoparticles comprise about 80-85% by weight of said first lipid, 15-20% by weight of said second lipid and about 2-5% of the surfactant by weight of said lipid nanoparticles.

24. A composition according to claim 23, wherein said first lipid is cetylpalmitate and said second lipid is caprylic/capric triglyceride.

25. A composition according to claim 18, wherein the vitamin D derivative is selected from calcipotriol, calcitriol, maxacalcitol or tacalcitol.

26. A composition according to claim 25, wherein the vitamin D derivative is calcipotriol or calcipotriol monohydrate.

27. A composition according to claim 18, wherein the lipid nanoparticles have a mean/average particle size/diameter in the range of about 10-800 nm, in particular about 50-600 nm, such as about 100-500 nm.

28. A composition according to claim 18, wherein the lipid nanoparticles are present in an amount of 1-40% by weight, such as about 5-30% by weight or about 10-20% by weight, of the composition.

29. A composition according to claim 18 further comprising an alkaline buffer such that the pH of the composition is 7.5 or more.

30. A composition according to claim 29, wherein the alkaline buffer is an amine with lipid anchoring.

31. A composition according to claim 30, wherein the amine is selected from triethanolamine, trometamol, monoethanolamine or diethanolamine.

32. A composition according to claim 18 further comprising an aqueous phase.

33. A composition according to claim 12, wherein the aqueous phase further comprises an emollient such as silicone oil, liquid paraffin or cholesterol.

34. A composition according to claim 33, wherein the emollient is included in an amount of about 10-50% by weight, or about 20-40% by weight, or about 30% by weight of the composition.

35. A composition according to claim 18 further comprising a thickener.

36. A composition according to claim 35, wherein the thickener is a carbomer, or a cellulose derivative, such as hydroxypropylcellulose, hydroxypropylmethylcellulose, hydroxyethylcellulose.

37. A composition according to claim 35, wherein the thickener is present in an amount of 0.1-5% by weight, in particular about 0.5% by weight, of the composition.

38. A method of preparing a composition according to claim 18, the method comprising
(a) solubilizing or dispersing a vitamin D derivative in an lipid phase comprising about 60-92% by weight of a first lipid with a melting point above body temperature, said first lipid being a wax selected from the group consisting of esters of C₁₂₋₂₄ alcohols and C₁₂₋₂₄ fatty acids, glyceryl triesters of C₁₂₋₂₄ fatty acids with an acid value of 0.1 or less, C₁₂₋₂₄ fatty alcohols, and cholesterol, and optionally about 1-40% by weight of a second lipid which is an oil at room temperature and miscible with the first lipid, said first lipid being in a molten state,
(b) dispersing the lipid phase obtained in step (a) in an aqueous phase comprising 0.2-10% by weight of a pharmaceutically acceptable surfactant selected from a poloxamer or an ethoxylated fatty alcohol,
(c) homogenizing the emulsion obtained in step (b) using a high-pressure homogenizer to form nanoparticles of the lipid phase comprising the vitamin D derivative and
(d) cooling the homogenized emulsion obtained in step (c) to solidify the nanoparticles.

39. A composition according to claim 18 for the prevention or treatment of an inflammatory or hyperproliferative skin condition such as psoriasis, sebopsoriasis, pustulosis palmoplantaris, dermatitis, ichtyosis, rosacea, acne or actinic keratosis.

40. A method of targeting a vitamin D derivative to the sebaceous gland of hair follicles, the method comprising applying on a skin area of a patient in need of such treatment, a therapeutically effective amount of a lipid nanoparticle composition according to claim 18.

41. The method of claim 40, wherein the area of skin in need of treatment comprises a comedone, pustule, papule or cyst associated with acne or rosacea.

42. The method of claim 40, wherein the vitamin D derivative is calcipotriol or calcpotriol monohydrate.

43. A pharmaceutical composition comprising a mixture of lipid nanoparticles according to claim 1 in a pharmaceutically acceptable aqueous vehicle.

44. A composition according to claim 43 further comprising an antioxidant.

45. A composition according to claim 44, wherein the antioxidant is selected from the group of BHA and BHT, or a mixture of BHA and BHT.
